(19) United States
(12) Patent Application Publication
(10) Pub. No.: US 2006/0188964 A1
(43) Pub. Date: Aug. 24, 2006

(54) Processing for producing and crystallizing G-protein coupled receptors

(76) Inventors: Filippo Mancia, New York, NY (US); Wayne Hendrickson, New York, NY (US); Paul Lee, New York, NY (US); Richard Axel, New York, NY (US)

Correspondence Address: COOPER & DUNHAM, LLP, 1185 AVENUE OF THE AMERICAS, NEW YORK, NY, 10036, US

(21) Appl. No.: 11/193,117

(22) Filed: Jul. 28, 2005

Related U.S. Application Data
(60) Provisional application No. 60/592,056, filed on Jul. 28, 2004.

(51) Int. Cl. C07K 14/705; C07H 21/04; C12P 21/04; C12N 15/63

(52) U.S. Cl. 435/69.7; 435/252.3; 435/320.1; 435/325; 530/350; 536/23.5

(57) ABSTRACT

This invention provides methods for producing a membrane-bound protein in mammalian cells. This invention also provides nucleic acids for making novel fusion proteins (e.g., GPCR fusion proteins). This invention further provides related bacterial expression vectors; expression methods; fusion proteins; bacterial cells; GPCR vector screens; bacterial spheroplasts; methods for making anti-GPCR antibodies; and GPCR binding screens. This invention also provides a method for identifying a reagent in which a membrane protein is likely to crystallize. Finally, this invention provides methods for producing crystals of a protein which, in a cell, is a membrane-bound protein.

This application claims the benefit of U.S. Provisional Application No. 60/592,056 filed Jul. 28, 2004, the contents of which are hereby incorporated by reference.

This invention was made with funding from the National Institutes of Health under grant numbers GM68671, GM68671, GM62529, R01-DK55758 and T32-GM97288. Accordingly, the United States Government has certain rights in this invention.

Throughout this application, various publications are referenced by author and date. Full citations for these publications may be found at the end of the specification preceding the claims. The disclosures of these publications in their entireties are hereby incorporated by reference into this application in order to more fully describe the state of the art as known to those skilled therein as of the date of the invention claimed herein.

### BACKGROUND OF THE INVENTION

The guanine nucleotide-binding protein (“G-protein”) coupled receptor (“GPCR”) superfamily is one of the most diverse groups of proteins. GPCRs comprise membrane proteins involved in a wide range of physiological signaling processes, and are attractive targets for pharmacological intervention to modify these processes in normal and pathological states.

GPCRs activate signaling paths in response to stimuli such as Ca²⁺, amines, hormones, neurotransmitters, peptides (and even large proteins), chemokines, and sensory stimuli. For example, some GPCRs are involved in the receptors found in the tongue (i.e. affecting taste) and nose (affecting smell). Some GPCRs are involved in regulating heartbeat, and some GPCRs are opiate receptors in the brain which affect one's predisposition to drug addiction.

GPCRs share many structural features. For example, GPCRs share a transmembrane structural motif comprising seven a helices connected by six loops of varying lengths. Binding of specific ligands to the seven a-helical transmembrane domains of GPCRs causes conformational changes that act as a switch to signal a G-protein, which in turn evoke subsequent intracellular responses. Many studies have been conducted to develop an understanding of the precise conformational transformation of an inactive GPCR into an activated form capable of interacting with a G-protein, in order to elucidate the molecular steps of cell surface activated receptor-mediated intracellular signaling. However, a high-resolution visualization of the entire GPCR structure is needed to understand the mechanism of GPCR signal transduction.

For these reasons and others, it is desirable to develop an atomic-level understanding of transmembrane signal transduction by GPCRs. For example, there is a need for structures for receptors stabilized in various relevant states, as complexes with natural signaling ligands, with pharmacological agonists and antagonists, and with signaling partner proteins, notably heterotrimeric G ligand complexes. X-ray crystallography could in principle be used to determine structures of GPCRs at the desired resolution of detail.

However, determining the structure of a GPCR is a challenging task. Crystallization of GPCRs for X-ray diffraction studies is difficult because GPCRs are membrane proteins. In addition, most GPCRs are found naturally only in very small quantities. Purification of GPCR proteins from natural sources can be difficult and time consuming, and the amount of purified protein is often too small for structural studies and functional characterizations.

Many barriers remain for production, through conventional techniques, of membrane proteins at levels of abundance and quality suitable for structural determinations, and this is particularly true for eukaryotic proteins. All of the structures determined to date for eukaryotic membrane proteins have come from naturally abundant sources. An example of such abundant protein is bovine rhodopsin, which is the only GPCR whose structure has been resolved at the atomic level.

### SUMMARY OF THE INVENTION

This invention provides a method for producing a membrane-bound protein in high yield, which comprises the steps of (a) culturing a mammalian cell and progeny thereof having therein an expression vector which coordinately expresses both (i) the membrane-bound protein and (ii) a luminescent protein, under conditions permitting selection of cells expressing the luminescent protein; (b) selecting cells cultured in step (a) which express a high yield of the luminescent protein so as to thereby select cells expressing a high yield of the membrane-bound protein; and (c) treating the cells selected in step (b) so as to recover therefrom the membrane-bound protein in high yield.

This invention also provides a first nucleic acid encoding a fusion protein comprising consecutive amino acids, the amino acid sequence of which corresponds to the amino acid sequence of a serotonin receptor and immediately contacting thereto the amino acid sequence of a targeting polypeptide which, upon expression of the fusion protein in a bacterium, causes the fusion protein so expressed to become situated in the bacterium's periplasmic space with the hydrophobic portion thereof being membrane-bound.

This invention further provides a second nucleic acid encoding a fusion protein comprising (a) a G protein coupled receptor (GPCR) and (b) a targeting polypeptide which, upon expression of the fusion protein in a bacterium, causes the fusion protein so expressed to become situated in the bacterium's periplasmic space with the hydrophobic portion thereof being membrane-bound.

This invention still further provides a third nucleic acid encoding a fusion protein comprising (i) a G protein coupled receptor (GPCR), (ii) a bacterial signal peptide, and (iii) a targeting polypeptide which, upon expression of the fusion protein in a bacterium, causes the fusion protein so expressed to become situated to the bacterium's periplasmic space with the hydrophobic portion thereof being membrane-bound.

This invention further provides a first, second and third bacterial expression vector comprising the first, second and third nucleic acid, respectively.

This invention further provides a method for producing a membrane-bound protein in high yield, which comprises the steps of (a) culturing a bacterial cell and progeny thereof having therein an expression vector which coordinately expresses both (i) the membrane-bound protein and (ii) a luminescent protein, under conditions permitting selection of cells expressing the luminescent protein; (b) selecting cells cultured in step (a) which express a high yield of the luminescent protein so as to thereby select cells expressing a high yield of the membrane-bound protein; and (c) treating the cells selected in step (b) so as to recover therefrom the membrane-bound protein in high yield.

This invention also provides a method for expressing a G protein coupled receptor (GPCR) in a bacterial cell comprising culturing a bacterial cell comprising the second or third expression vector.

This invention also provides a first fusion protein comprising serotonin receptor and a targeting protein which, upon the fusion protein's expression in a bacterium, causes the fusion protein to be directed to the bacterium's periplasmic space with the hydrophobic portion thereof remaining membrane-bound.

This invention further provides a second fusion protein comprising (a) a non-glycosylated G protein coupled receptor (GPCR) which binds to the ligand to which the glycosylated form of the GPCR binds, and (b) a targeting protein which, upon the fusion protein's expression in a bacterium, causes the fusion protein to be directed to the bacterium's periplasmic space with the hydrophobic portion thereof remaining membrane-bound.

This invention still further provides a third fusion protein comprising (i) a G protein coupled receptor (GPCR), (ii) a bacterial signal peptide, and (iii) a targeting protein which, upon the fusion protein's expression in a bacterium, causes the fusion protein to be directed to the bacterium's periplasmic space.

This invention provides a first, second and third bacterial cell comprising the first, second and third expression vector, respectively.

This invention also provides a method for determining which vector(s) among a plurality of G protein coupled receptor (GPCR)-encoding bacterial expression vectors give rise to a desired level of GPCR expression in bacteria comprising (a) culturing a plurality of populations of bacteria, wherein (i) each population is transfected with the second or third expression vector, (ii) each population of bacteria is comprised of the same strain as the others, and (iii) each population of bacteria is transfected with a different vector than are the other populations, and (b) determining which population(s) express the desired level of GPCR, thereby determining which expression vectors give rise to a desired level of GPCR expression.

This invention further provides a method for producing a bacterial spheroplast having a G protein coupled receptor (GPCR) affixed to the outer membrane thereof comprising (a) culturing the second or third bacterial cells, and (b) removing the outer cell membranes thereof.

This invention still further provides a bacterial spheroplast having a G protein coupled receptor (GPCR) affixed to its outer membrane.

This invention still further provides a method for determining whether an agent binds to a G protein coupled receptor (GPCR) comprising (a) contacting the agent with a bacterial spheroplast having the GPCR affixed to its outer membrane under conditions permitting binding of the GPCR on the spheroplast to a known ligand thereof, and (b) determining whether the agent binds to the GPCR on the spheroplast, thereby determining whether the agent binds to the GPCR.

This invention also provides a method for producing an antibody against a G protein coupled receptor (GPCR) comprising administering to a mammalian subject a bacterial spheroplast having the GPCR affixed to its outer membrane, so as to cause production in the subject of an antibody against the GPCR.

This invention also provides a method for identifying a reagent in which a membrane protein is likely to crystallize, which protein is known to be soluble in a detergent which preserves the protein's structural integrity, comprising the steps of (a) permitting equilibration between a solution containing the detergent at a predetermined concentration and a matrix of precipitant-containing reagents, wherein (i) the reagents collectively comprise a plurality of precipitant types and/or concentrations, and (ii) each reagent contains only one precipitant at one concentration; and (b) after equilibration occurs, identifying one of the equilibrated reagents, if any, in which cloud point has been achieved, such equilibrated reagent being one in which the membrane protein is likely to crystallize.

This invention also provides a method for producing crystals of a protein which, in a cell, is membrane-bound, comprising the steps of (a) identifying a reagent in which the membrane protein is likely to crystallize according to the instant method, and (b) growing crystals of the protein in the reagent identified in step (a).

This invention also provides a method for producing crystals of a protein which, in a cell, is a membrane-bound protein which comprises the steps of (a) producing the protein in high yield according to the instant mammalian cell-based method, and (b) treating the protein from step (a) so as to form crystals thereof.

This invention also provides a method for producing crystals of a protein which, in a cell, is a membrane-bound protein which comprises the steps of (a) producing the protein in high yield according to the instant mammalian cell-based method; and (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i).

This invention also provides a method for producing crystals of a protein which, in a cell, is a membrane-bound protein which comprises the steps of (a) producing the protein in high yield according to the instant bacterial cell-based method; and (b) treating the protein from step (a) so as to form crystals thereof.

This invention also provides a method for producing crystals of a protein which, in a cell, is a membrane-bound protein which comprises the steps of (a) producing the protein in high yield according to the instant bacterial cell-based method; and (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i).

This invention also provides a method for producing, and obtaining the crystal structure of, a protein which, in a cell, is a membrane-bound protein which comprises the steps of (a) producing the protein in high yield according to the instant mammalian cell-based method; (b) treating the protein from step (a) so as to form crystals thereof; and (c) obtaining a crystal structure for the crystals formed in step (b).

This invention also provides a method for producing crystals of a protein which, in a cell, is a membrane-bound protein which comprises the steps of (a) producing the protein in high yield according to the instant mammalian cell-based method; (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i); and
   (c) obtaining a crystal structure for the crystals formed in step (b).

This invention also provides a method for producing crystals of a protein which, in a cell, is a membrane-bound protein which comprises the steps of (a) producing the protein in high yield according to the instant bacterial cell-based method; (b) treating the protein from step (a) so as to form crystals thereof; and (c) obtaining a crystal structure for the crystals formed in step (b).

Finally, this invention provides a method for producing crystals of a protein which, in a cell, is a membrane-bound protein which comprises the steps of (a) producing the protein in high yield according to the instant bacterial cell-based method; (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i); and
   (c) obtaining a crystal structure for the crystals formed in step (b).

### BRIEF DESCRIPTION OF THE FIGURES

FIG. 1: Flow chart corresponding to a method of amplified expression of a functional G-protein coupled receptor, according to one embodiment.

FIG. 2: Flow chart corresponding to a method of amplified expression of a functional G-protein coupled receptor, according to another embodiment.

FIG. 3: Flow chart corresponding to a method of amplified expression of a functional serotonin receptor, according to one embodiment.

FIG. 4: Flow chart corresponding to a method of amplified expression of a 5HT2c receptor, according to one embodiment.

FIG. 5A: Flow chart corresponding to a method of amplified expression of a functional G-protein coupled receptor, according to another embodiment.

FIG. 5B: Flow chart corresponding to a method of amplified expression of a functional serotonin receptor, according to another embodiment.

FIG. 6: A model of G-protein coupling of GPCR activation to effector targets.

FIG. 7: A table comparing characteristics of serotonin receptors.

FIG. 8: A comparison of selected 5HT receptors.

FIG. 9: Amino-acid sequence and transmembrane topology of the rat 5HT2c serotonin receptor.

FIG. 10A: Stereodiagram of an approximate model of the serotonin receptor which is based on the alpha-carbon template of Baldwin et al. (1997), but inverted from the rhodopsin convention to the cytoplasm-down orientation more commonly used for GPCRs and in particular for the serotonin receptor shown in FIG. 3. Extramembranous portions drawn in rough proportion to the length or mass of these segments.

FIG. 10B: Ribbon diagram of bovine rhodopsin.

FIG. 11: Flow chart corresponding to a method of amplified expression of a functional G-protein coupled receptor, according to another embodiment.

FIG. 12: Flow cytometry sorting of GFP-serotonin receptor expressing HEK 293 cells. The populations are represented progressively darker in accordance with increasing levels of GFP expression.

FIG. 13: Western blot analysis of cells at different stages of selection.

FIGS. 14A and 14B: Ligand binding to membranes isolated from 293 cells enriched for the expression of serotonin-receptor. Saturation curve for tritiated LSD (FIG. 14A). Scatchard plot of data shown in FIG. 14A (FIG. 14B).

FIG. 15: Western blot probed with anti-5HT2c antibody in which cells of stable 293 cell line expressing the 5HT2c receptor were run on an SDS-PAGE gel, without and with the addition of a deglycosylation enzyme.

FIG. 16: Western blot analysis of cells transfected with glycosylation-site mutants of the 5HT2c receptor.

FIG. 17: Western blot analysis of individual clones generated from cells expressing 5HT2c receptor mutated at three sites, N39D, N204D and N205D.

FIG. 18: Flow chart corresponding to a method of amplified expression of a functional G-protein coupled receptor, according to another embodiment.

FIGS. 19A and 19B: Data from a typical experiment with expression of various MBP-5HT2c fusion constructs. FIG. 19A shows a western blot probed with anti 5HT2c antibody. FIG. 19B shows relative specific activity for each construct.

FIGS. 20A and 20B: Ligand binding to bacterial spheroplasts isolated from E. coli cells expressing the MBP-serotonin receptor fusion protein. Saturation curve for tritiated mesulergine (FIG. 20A). Scatchard plot of data shown in FIG. 20A (FIG. 20B).

FIGS. 21A and 21B: Expression data and relative activity data for the 5HT1a receptor. FIG. 21A shows a western blot probed with anti-MBP polyclonal antibody (New England Biolabs). FIG. 21B shows ligand binding assays performed on spheroplasts.

FIG. 22: Western blots corresponding to expression data for the 5HT1b and 5HT7.

FIGS. 23A and 23B: Results from testing different maltoside detergents which show yield and activity of 5HT2c receptor solubilized by maltoside detergents. FIG. 23A shows a western blot probed with anti-5HT2c antibody. FIG. 23B shows a specific activity measured at 10 nM ³H-LSD.

FIG. 24: Affinity purification of 5HT2c receptor. FIG. 24 shows a western blot performed on fractions collected at various stages of purification, and probed with anti-5HT2c antibody.

FIG. 25: Analysis of the purified 5HT2c. A Coomassie-stained SDS-PAGE gel of purified material from the same preparation.

FIG. 26: Comparison of the activity of the 5HT2c receptor solubilized in different detergents.

FIGS. 27A and 27B: Gel electrophoresis of purified MBP-receptor fusion protein. Each gel is stained by Coomassie blue to quantify protein. Denaturing polyacrylamide-SDS gel compared with molecular mass standards and bovine serum albumin (BSA) concentration standards (FIG. 27A). Native polyacrylamide gel compared with BSA. Each shows a single, sharp band indicative of homogeneity and purity (FIG. 27B).

FIGS. 28A and 28B: Silver-stained denaturing gel of fractions collected from the rerun of the 150 kDa species, and the corresponding activity profile, respectively. FIG. 28A shows silver stained denaturing gel of MBP-5HT2c. FIG. 28B shows activity profile of peak fractions.

FIGS. 29A and 29B: Expression and activity of C-terminal fusions to 5HT2c. FIG. 29A shows quantitative western blot analysis of MBP-5HT2c-Gαq (lanes 3 to 7) and MBP-5HT2c-GαiqC fusions (lanes 8 to 12) compared to MBP-5HT2c (lane 1) and MBP-5HT2c-TRX (lane 2). FIG. 29B shows relative specific activity data, measured at 2 nM 3H-LSD with and without 10 mM mesulergine.

FIG. 30: Crystals of the MBP-serotonin receptor fusion protein. Typical crystals of this kind have dimensions of 80 μm×80 μm×30 μm.

FIG. 31: Diffraction pattern of a crystal obtained using the PF6 screen.

FIGS. 32A and 32B: Expression of olfactory receptor SP1. FIG. 32A shows a western blot probed with anti-MBP antibody. FIG. 32B shows a western blot probed with anti-SP1 antibody.

FIG. 33: Detergent solubilization of olfactory receptor SP1.

FIG. 34: Schematic representation of the GFP-selection mammalian expression system. (A) The expression vector pFM-1.1. The protein of interest is placed downstream from the strong constitutive CMV promoter. Following the termination codon of the protein of interest is an internal ribosome entry site (IRES) which enables translation of GFP to be initiated from an internal site of the bicistronic mRNA transcript. This enables production of two separate proteins: GFP, and the protein of interest. Similar vectors are now commercially available (for example, pIRES-GFP from Clontech, Inc.) pFM-1.2 differs from pFM-1.1 in that it contains an antibiotic resistance gene for puromycin under control of a separate promoter. The pFM vectors are based on a pBluescript parent vector, which was modified by the insertion of the CMV promoter region, followed by a multiple cloning site, and an IRES-GFP segment which included a appropriate poly-A tail. (B) Enrichment Procedure. A highly-expressing cell line is developed by repeated rounds of cell sorting, selecting for the highest levels of GFP-derived fluorescence. Since both GFP and the protein of interest are expressed from the same mRNA, GFP fluorescence provides a useful surrogate correlated to levels of the protein of interest.

FIG. 35: Expression of the Serotonin receptor using the GFP selection method. (A) Flow cytometry sorting of GFP-serotonin receptor expressing HEK-293T cells. The populations are represented progressively darker in accordance with increasing levels of GFP expression. (B) Western blot analysis of cells at different stages of selection. Lane 1 represents cells 48 hours after transfection; lane 2 represents cells after puromycin selection; lane 3 represents cells after GFP selection; lane 4 represents untransfected cells. 20,000 cells were run on each lane, and the samples were deglycosylated for 1 hr on ice with endoglycosidase F prior to loading. The membrane was probed with an anti-5HT2c rabbit polyclonal antibody generously provided by Dr Jon Backstrom [26]. (C) Ligand binding to membranes isolated from HEK-293T cells enriched for the expression of serotonin-receptor. (i) Saturation curve for tritiated lysergic acid diamine (LSD). (ii) Scatchard plot of data shown in panel (i).

FIG. 36: Expression of the secreted protein resistin using the GFP-selection method. (A) Flow cytometry sorting of Resistin/GFP expressing HEK-293T cells. Five sequential cell sorting runs were performed in accordance with the scheme shown in FIG. 1B. Fluorescence traces are shown for cells from each sorting run. After each sort, the top 0.6% of the most fluorescent cells were pooled and expanded. With each sort, the average fluorescence per cell increases, until reaching a plateau at sort 5. (B) Resistin protein levels monitored by Coomassie-blue staining of SDS gels of cell culture supernatants. Resistin levels of cell supernatants from each sort increase in concordance with GFP fluorescence. Each of the three lanes represents equivalent samples taken from three independently plated culture dishes from each sort, showing the reproducible nature of the increase in protein production. Only the section of the gel corresponding to resistin is shown. The lower two panels show similar analyses of supernatants from cells transiently transfected using either calcium phosphate or Effectene (Qiagen, Inc.). Each lane was loaded with cell supernatant concentrated by a 60% ammonium sulfate cut, which is known to precipitate resistin. The load of each lane corresponds to ˜300 μl of conditioned serum-free medium. Cells were grown in 75 mm dishes with 10 ml medium per dish; sorted cells were transferred to serum-free media at 80% confluence, and conditioned medium was collected after three days. For transiently transfected cells, transfection was performed at 80% confluence, and the media was changed to serum free medium 24 hours post-transfection. Supernatants were collected after 3 days, and treated as above for gel analysis.

### DETAILED DESCRIPTION OF THE INVENTION

Definitions

“Cloud point”, as used in the field of X-ray crystallography, means the precipitant concentration above which a soluble protein in solution becomes insoluble, and below which a soluble protein in solution remains soluble.

“Eukaryotic cell” means any cell with a true nucleus bounded by a nuclear envelope. Eukaryotic cells include, for example, animal cells (e.g., mammalian cells) and plant cells.

“Expression” means the cellular production of protein encoded by a particular nucleic acid. Expression includes, for example, transcription of DNA, processing of the resulting mRNA product and its translation into an active protein (see Sambrook et al. 1989).

“Expression vector” shall mean a nucleic acid encoding a nucleic acid of interest and/or a protein of interest, which nucleic acid, when placed in a cell, permits the expression of the nucleic acid or protein of interest. Expression vectors are well known in the art.

“Fusion protein” means a protein having a single polypeptide chain, which chain comprises two or more moieties which in nature do not exist as part of the same polypeptide chain. Examples of fusion proteins include a polypeptide chain comprising GPCR and MBP, wherein the GPCR and MBP are either contiguous or separated by a linker region.

“Functional GPCR” means an expression product which is effective as a receptor of the associated G-protein. As used herein, “GPCR” and “functional GPCR” are synonymous, unless otherwise indicated.

“GPCR” means G-protein coupled receptor. GPCRs include, without limitation, serotonin olfactory receptors, glycoprotein hormone receptors, chemokine receptors, adenosine receptors, biogenic amine receptors, melanocortin receptors, neuropeptide receptors, chemotactic receptors, somatostatin receptors, opioid receptors, melatonin receptors, calcitonin receptors, PTH/PTHrP receptors, glucagon receptors, secretin receptors, latrotoxin receptors, metabotropic glutamate receptors, calcium receptors, GABA-B receptors, pheromone receptors, and other G-protein coupled, seven-transmembrane segment receptors. In one embodiment, the GPCR has a loop deletion, or an N- and/or C-terminal truncation.

“GFP” means green fluorescent protein. GFP is a protein produced by the jellyfish Aequorea victoria which fluoresces bright green upon exposure to ultraviolet or blue light.

“Isolated” membrane-bound protein includes, for example, protein in isolated bilayers (either as naturally enriched or reconstituted), and in detergent micelles.

“Likely” to crystallize, with respect to a protein solubilized in a first reagent, means more likely to crystallize in the first reagent than in a second reagent.

“Luminescent protein” means any protein which gives off visible light upon exposure to ultraviolet or visible light (e.g., GFP).

“Mammalian cell” shall mean any mammalian cell. Mammalian cells include, without limitation, cells which are normal, abnormal and transformed, and are exemplified by neurons, epithelial cells, muscle cells, blood cells, immune cells, stem cells, osteocytes, endothelial cells and blast cells. Examples of mammalian cells commonly used for protein expression include HEK 293 cells, NIH 3T3 cells, CHO cells and TOF cells.

“Matrix” of reagents means a plurality of reagents in separate compartments. In one embodiment, the reagents are contained within a single apparatus. In another embodiment, the reagents are contained in a plurality of apparati. Apparati envisioned for this purpose include, without limitation, standard crystallization plates, and plates and scaffolds used in microassays and high-throughput screening.

“MBP” shall mean maltose-binding protein.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“PEG” means polyethylene glycol.

“PEG-related compound” means glycerol, ethylene glycol, or a derivative of PEG, such as PEG monomethyl ether or PEG dimethyl ether.

“Precipitant” means an agent which, at a high enough concentration, causes a solubilized protein to become insoluble. Precipitants include, for example, PEG, PEG-related compounds, salts, and small volatile organic compounds.

“Polypeptide” and “protein” are used equivalently, and each means a polymer of amino acid residues. The amino acid residues can be naturally occurring or chemical analogues thereof. Polypeptides and proteins can also include modifications such as glycosylation, lipid attachment, sulfation, hydroxylation, and ADP-ribosylation.

“Protein cleavage site” means a site recognized and cleaved by a site-specific protease, such as TEV protease which recognizes and cleaves the site ENLYFQ·GS.

“Stabilize”, with respect to a protein, means to inhibit the protein's degradation or any other physical modification which adversely affects its function.

### EMBODIMENTS OF THE INVENTION

The present disclosure describes methodologies for expressing, purifying, characterizing and crystallizing functional GPCRs.

Specifically, this invention provides a method for producing a membrane-bound protein in high yield, which comprises the steps of (a) culturing a mammalian cell and progeny thereof having therein an expression vector which coordinately expresses both (i) the membrane-bound protein and (ii) a luminescent protein, under conditions permitting selection of cells expressing the luminescent protein; (b) selecting cells cultured in step (a) which express a high yield of the luminescent protein so as to thereby select cells expressing a high yield of the membrane-bound protein; and (c) treating the cells selected in step (b) so as to recover therefrom the membrane-bound protein in high yield.

In one embodiment, the membrane-bound protein is a G protein coupled receptor (GPCR), such as a human GPCR. In another embodiment, the luminescent protein is green fluorescent protein (GFP). “Mammal” shall include, without limitation, a human, non-human primate, mouse, rat, guinea pig or rabbit.

In another embodiment, the instant method further comprises repeating steps (a) and (b) prior to step (c). In a further embodiment of the instant method, the vector further encodes a protein conferring resistance to an antibiotic, and the conditions permitting selection of cells expressing the luminescent protein encoded by the vector comprise the presence of the antibiotic in a medium in which the cells are cultured. “Antibiotic” includes, without limitation, ampicillin, kanamycin, chloramphenicol and tetracycline. In one embodiment of the instant method, the cells selected in step (b) have an average of at least 3 million copies of the membrane-bound protein per cell. In another embodiment, the cells selected in step (b) have an average of at least 5 million copies of the membrane-bound protein per cell. In a further embodiment, the cells selected in step (b) have an average of at least 10 million copies of the membrane-bound protein per cell.

This invention also provides a first nucleic acid encoding a fusion protein comprising a serotonin receptor and a targeting polypeptide which, upon expression of the fusion protein in a bacterium, causes the fusion protein so expressed to become situated in the bacterium's periplasmic space with the hydrophobic portion thereof being membrane-bound.

In one embodiment of the first nucleic acid, the serotonin receptor is human serotonin receptor. In another embodiment, the targeting polypeptide is maltose binding protein (MBP). In a further embodiment, the bacterium is E. coli. “Bacterium” includes, without limitation, E. coli and B. subtilis.

In another embodiment of the first nucleic acid, the fusion protein further comprises a first linker region between the serotonin receptor and the targeting polypeptide. In a further embodiment, the first linker region comprises a protein cleavage site and/or an affinity purification tag. “Affinity purification tag” includes, without limitation, biotin, poly-histidine, streptavidin-binding peptides and antibody tags.

In a further embodiment, the fusion protein further comprises a protein which stabilizes the serotonin receptor, such as Gα protein.

In a further embodiment of the first nucleic acid, the fusion protein further comprises a second linker region between the serotonin receptor and the polypeptide which stabilizes it. In one embodiment, the second linker region comprises a protein cleavage site.

This invention further provides a second nucleic acid encoding a fusion protein comprising (a) a G protein coupled receptor (GPCR) and (b) a targeting polypeptide which, upon expression of the fusion protein in a bacterium, causes the fusion protein so expressed to become situated in the bacterium's periplasmic space with the hydrophobic portion thereof being membrane-bound.

In one embodiment of the second nucleic acid, the GPCR is a human GPCR. In another embodiment, the targeting polypeptide is maltose binding protein (MBP). In a further embodiment, the bacterium is E. coli.

In another embodiment of the second nucleic acid, the fusion protein further comprises a first linker region between the GPCR and the targeting polypeptide. In one embodiment, the first linker region comprises a protein cleavage site and/or an affinity purification tag.

In a further embodiment of the second nucleic acid, the fusion protein further comprises a polypeptide which stabilizes the GPCR, such as Gα protein.

In a further embodiment of the second nucleic acid, the fusion protein further comprises a second linker region between the GPCR and the polypeptide which stabilizes it. In one embodiment, the second linker region comprises a protein cleavage site.

This invention still further provides a third nucleic acid encoding a fusion protein comprising (i) a G protein coupled receptor (GPCR), (ii) a bacterial signal peptide, and (iii) a targeting polypeptide which, upon expression of the fusion protein in a bacterium, causes the fusion protein so expressed to become situated to the bacterium's periplasmic space with the hydrophobic portion thereof being membrane-bound.

In one embodiment of the third nucleic acid, the GPCR is a human GPCR. In another embodiment, the targeting polypeptide is maltose binding protein (MBP). In a further embodiment, the bacterium is E. coli.

In another embodiment of the third nucleic acid, the fusion protein further comprises a first linker region between the GPCR and the targeting polypeptide. In one embodiment, the first linker region comprises a protein cleavage site and/or an affinity purification tag.

In a further embodiment of the third nucleic acid, the fusion protein further comprises a protein which stabilizes the GPCR, such as Gα protein. In another embodiment, the fusion protein further comprises a second linker region between the GPCR and the protein which stabilizes it. In one embodiment, the second linker region comprises a protein cleavage site.

In a further embodiment of the third nucleic acid, the fusion protein further comprises a third linker region between the signal peptide and the targeting polypeptide. In one embodiment, the third linker region comprises an affinity purification tag and/or a detection tag. “Detection tag” includes, without limitation, poly-histidine, an antibody, and a streptavidin-binding peptide.

This invention further provides a first, second and third bacterial expression vector comprising the first, second and third nucleic acid, respectively. In one embodiment, the vector is a vector for expression in E. coli.

This invention also provides a first method for expressing serotonin receptor in a bacterial cell comprising culturing a bacterial cell comprising the first expression vector. In one embodiment, the bacterial cell is E. coli.

This invention further provides a method for producing a membrane-bound protein in high yield, which comprises the steps of (a) culturing a bacterial cell and progeny thereof having therein an expression vector which coordinately expresses both (i) the membrane-bound protein and (ii) a luminescent protein, under conditions permitting selection of cells expressing the luminescent protein; (b) selecting cells cultured in step (a) which express a high yield of the luminescent protein so as to thereby select cells expressing a high yield of the membrane-bound protein; and (c) treating the cells selected in step (b) so as to recover therefrom the membrane-bound protein in high yield.

This invention also provides a second method for expressing a G protein coupled receptor (GPCR) in a bacterial cell comprising culturing a bacterial cell comprising the second or third expression vector. In one embodiment, the bacterial cell is E. coli. In another embodiment, the GPCR is a human GPCR.

This invention also provides a first fusion protein comprising serotonin receptor and a targeting protein which, upon the fusion protein's expression in a bacterium, causes the fusion protein to be directed to the bacterium's periplasmic space with the hydrophobic portion thereof remaining membrane-bound.

This invention further provides a second fusion protein comprising (a) a non-glycosylated G protein coupled receptor (GPCR) which binds to the ligand to which the glycosylated form of the GPCR binds, and (b) a targeting protein which, upon the fusion protein's expression in a bacterium, causes the fusion protein to be directed to the bacterium's periplasmic space with the hydrophobic portion thereof remaining membrane-bound.

This invention still further provides a third fusion protein comprising (i) a G protein coupled receptor (GPCR), (ii) a bacterial signal peptide, and (iii) a targeting protein which, upon the fusion protein's expression in a bacterium, causes the fusion protein to be directed to the bacterium's periplasmic space.

This invention provides a first, second and third bacterial cell comprising the first, second and third expression vector, respectively.

This invention also provides a method for determining which vector(s) among a plurality of G protein coupled receptor (GPCR)-encoding bacterial expression vectors give rise to a desired level of GPCR expression in bacteria comprising (a) culturing a plurality of populations of bacteria, wherein (i) each population is transfected with the second or third expression vector, (ii) each population of bacteria is comprised of the same strain as the others, and (iii) each population of bacteria is transfected with a different vector than are the other populations, and (b) determining which population(s) express the desired level of GPCR, thereby determining which expression vectors give rise to a desired level of GPCR expression.

This invention further provides a method for producing a bacterial spheroplast having a G protein coupled receptor (GPCR) affixed to the outer membrane thereof comprising (a) culturing the second or third bacterial cells, and (b) removing the outer cell membranes thereof.

This invention still further provides a bacterial spheroplast having a G protein coupled receptor (GPCR) affixed to its outer membrane. In one embodiment of the instant bacterial spheroplast, the spheroplast is an E. coli spheroplast. In another embodiment, the GPCR is a human GPCR.

This invention further provides a bacterial spheroplast produced by the instant method.

This invention still further provides a method for determining whether an agent binds to a G protein coupled receptor (GPCR) comprising (a) contacting the agent with a bacterial spheroplast having the GPCR affixed to its outer membrane under conditions permitting binding of the GPCR on the spheroplast to a known ligand thereof, and (b) determining whether the agent binds to the GPCR on the spheroplast, thereby determining whether the agent binds to the GPCR. In one embodiment, the agent is an antibody. “Antibody” includes, by way of example, both naturally occurring and non-naturally occurring antibodies. Specifically, this term includes polyclonal and monoclonal antibodies, and fragments thereof (e.g., Fab fragments). Furthermore, this term includes chimeric antibodies and wholly synthetic antibodies, and fragments thereof.

This invention also provides a method for producing an antibody against a G protein coupled receptor (GPCR) comprising administering to a mammalian subject a bacterial spheroplast having the GPCR affixed to its outer membrane, so as to cause production in the subject of an antibody against the GPCR.

This invention also provides a method for identifying a reagent in which a membrane protein is likely to crystallize, which protein is known to be soluble in a detergent which preserves the protein's structural integrity, comprising the steps of: (a) permitting equilibration between a solution containing the detergent at a predetermined concentration and a matrix of precipitant-containing reagents, wherein (i) the reagents collectively comprise a plurality of precipitant types and/or concentrations, and (ii) each reagent contains only one precipitant at one concentration; and (b) after equilibration occurs, identifying one of the equilibrated reagents, if any, in which cloud point has been achieved, such equilibrated reagent being one in which the membrane protein is likely to crystallize. “Detergent”, when used in the context of protein crystallization, includes, without limitation, SDS, alkyl maltopyranosides, alkyl glucopyranosides, alkyl dimethylamine-N-oxides, digitonin, and alkyl FOS-CHOLINEs.

In one embodiment, the membrane protein is a GPCR. In another embodiment, the solution containing the detergent further comprises a buffer at a predetermined concentration. In a further embodiment, the precipitant in each reagent is selected from the group consisting of a PEG-related compound, a salt, an organic solvent, and a small volatile organic compound. “Salt” includes, without limitation, NaCl, an NH₄⁺-containing salt, a Ca⁺⁺-containing salt and a Mg⁺⁺-containing salt. “Small volatile organic compound” includes, without limitation, MPD (2,4-methyl pentane diol), 1,6-hexane diol, and heptane triol.

In still a further embodiment, in step (a), the solution containing the detergent further comprises the membrane protein at a concentration and purity level suitable for crystallization, and in step (b) one of the equilibrated reagents, if any, in which protein precipitation has been achieved is identified, such equilibrated reagent being one in which the membrane protein is likely to crystallize. “Concentration and purity level” of protein sufficient for crystallization includes, for example, 0.5-40 mg/ml protein and 90-100% purity.

In another embodiment of this method, the method further comprises the steps of: (a) permitting equilibration between the equilibrated reagent identified in step (b) and a matrix of buffers, wherein (i) the buffers collectively have a plurality of pH's, and (ii) each buffer has only one pH; and (b) after a suitable period of time, identifying one of the equilibrated buffers, if any, in which protein precipitation or crystallization has been achieved, thereby identifying a reagent suitable for crystallizing the membrane protein. In a further embodiment, the method further comprises assessing the quality of any protein crystals formed.

This invention also provides a method for producing crystals of a protein which, in a cell, is membrane-bound, comprising the steps of (a) identifying a reagent in which the membrane protein is likely to crystallize according to the instant method, and (b) growing crystals of the protein in the reagent identified in step (a). In one embodiment, the protein is a G protein coupled receptor (GPCR).

This invention also provides a method for producing crystals of a protein which, in a cell, is a membrane-bound protein which comprises the steps of (a) producing the protein in high yield according to the instant mammalian cell-based method, and (b) treating the protein from step (a) so as to form crystals thereof.

This invention also provides a method for producing crystals of a protein which, in a cell, is a membrane-bound protein which comprises the steps of (a) producing the protein in high yield according to the instant mammalian cell-based method; and (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i).

This invention also provides a method for producing crystals of a protein which, in a cell, is a membrane-bound protein which comprises the steps of (a) producing the protein in high yield according to the instant bacterial cell-based method; and (b) treating the protein from step (a) so as to form crystals thereof.

This invention also provides a method for producing crystals of a protein which, in a cell, is a membrane-bound protein which comprises the steps of (a) producing the protein in high yield according to the instant bacterial cell-based method; and (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i).

This invention also provides a method for producing, and obtaining the crystal structure of, a protein which, in a cell, is a membrane-bound protein which comprises the steps of (a) producing the protein in high yield according to the instant mammalian cell-based method; (b) treating the protein from step (a) so as to form crystals thereof; and (c) obtaining a crystal structure for the crystals formed in step (b).

This invention also provides a method for producing crystals of a protein which, in a cell, is a membrane-bound protein which comprises the steps of (a) producing the protein in high yield according to the instant mammalian cell-based method; (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i); and
   (c) obtaining a crystal structure for the crystals formed in step (b).

This invention also provides a method for producing crystals of a protein which, in a cell, is a membrane-bound protein which comprises the steps of (a) producing the protein in high yield according to the instant bacterial cell-based method; (b) treating the protein from step (a) so as to form crystals thereof; and (c) obtaining a crystal structure for the crystals formed in step (b).

Finally, this invention provides a method for producing crystals of a protein which, in a cell, is a membrane-bound protein which comprises the steps of (a) producing the protein in high yield according to the instant bacterial cell-based method; (b) treating the protein from step (a) so as to form crystals thereof, wherein the treating comprises the steps of
(i) identifying a reagent in which the protein is likely to crystallize, which protein is known to be soluble in a detergent which preserves the protein's structural integrity, comprising the steps of (1) permitting equilibration between a solution containing the detergent at a predetermined concentration and a matrix of precipitant-containing reagents, wherein the reagents collectively comprise a plurality of precipitant types and/or concentrations, and each reagent contains only one precipitant at one concentration, and (2) after equilibration occurs, identifying one of the equilibrated reagents, if any, in which cloud point has been achieved, such equilibrated reagent being one in which the membrane protein is likely to crystallize, and
(ii) growing crystals of the protein in the reagent identified in step (i); and
   (c) obtaining a crystal structure for the crystals formed in step (b).

To facilitate an understanding of the material which follows, one may refer to Sambrook et al. (1989) for certain commonly used methodologies and/or terms which are not described in detail herein.

The following Exemplary Embodiments and Experimental Details are set forth to aid in an understanding of the subject matter of this disclosure, but are not intended to, and should not be construed to, limit in any way the claims which follow thereafter.

### EXEMPLARY EMBODIMENTS

Some embodiments are described exemplarily below to illustrate some methodologies contemplated by the subject disclosure. Many variations can be introduced on these embodiments without departing from the spirit of the disclosure or from the scope of the appended claims.

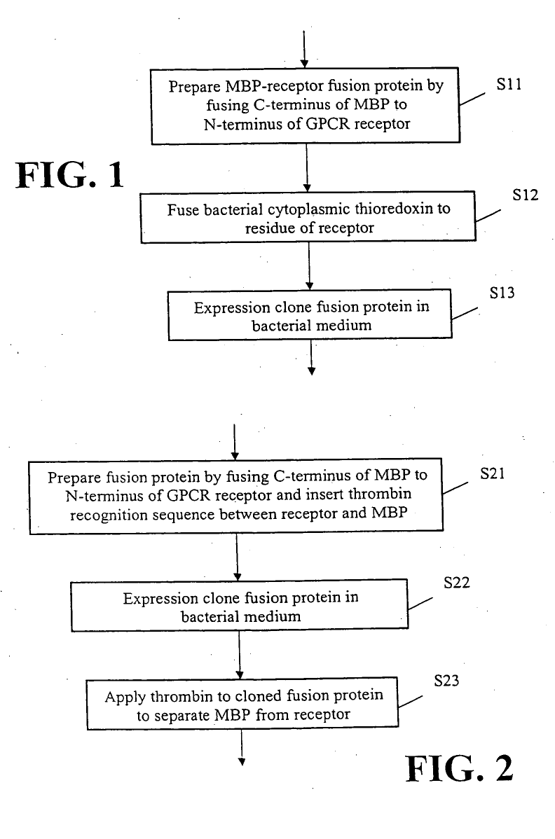

A method of amplified expression of a functional G-protein coupled receptor, according to one embodiment (FIG. 1), may comprise: (a) preparing an MBP-receptor fusion protein by fusing a C-terminus of a maltose-binding protein to an N-terminus of the G-protein coupled receptor without a signal sequence (step S11); (b) fusing bacterial cytoplasmic thioredoxin to a residue of the receptor (step S12); and (c) expression cloning the fusion protein in a bacterial medium (step S13). The method may further comprise inserting a thrombin-cleavable linkage between the maltose-binding protein and the G-protein coupled receptor, and adding thrombin to separate the maltose-binding protein and the G-protein coupled receptor after cloning. According to another embodiment, the method may further comprise inserting the MBP-receptor fusion protein in a bacterial membrane preparation, and extracting the fusion protein from the membrane preparation by applying a detergent system after cloning. The method may further comprise inserting additives in the bacterial expression to preserve activity of the G-protein coupled receptor.

A method of amplified expression of a functional G-protein coupled receptor, according to another embodiment (FIG. 2), may comprise: (a) preparing an MBP-receptor fusion protein by fusing a C-terminus of a maltose-binding protein to an N-terminus of the receptor without a signal sequence and insering a thrombin recognition sequence between the receptor and the maltose-binding protein (step S21); (b) expression cloning the fusion protein in a bacterial medium (step S22); and (c) applying thrombin to the cloned fusion protein to separate the maltose-binding protein from the receptor (step S23). The method may further comprise fusing bacterial cytoplasmic thioredoxin to a residue of the G-protein coupled receptor.

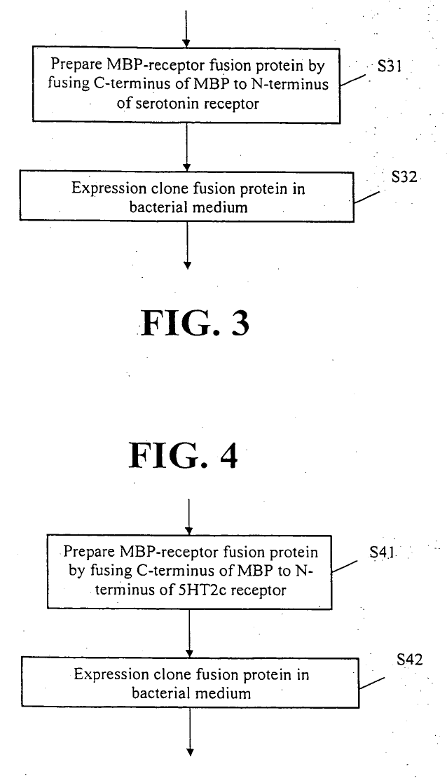

A method of amplified expression of a functional serotonin receptor, according to one embodiment (FIG. 3), may comprise: (a) preparing an MBP-receptor fusion protein by fusing a C-terminus of a maltose-binding protein to an N-terminus of the serotonin receptor without a signal sequence (step S31); and (b) expression cloning the fusion protein in a bacterial medium (step S32). The method may further comprise inserting a thrombin-cleavable linkage between the maltose-binding protein and the serotonin receptor. The method also may further comprise adding thrombin to separate the maltose-binding protein and the serotonin receptor after cloning. The method may further comprise fusing bacterial cytoplasmic thioredoxin to a residue of the serotonin receptor. According to another embodiment, the method may further comprise inserting the MBP-receptor fusion protein in a bacterial membrane preparation, and extracting the fusion protein from the membrane preparation by applying a detergent system after cloning. In addition, the method may further comprise inserting additives in the bacterial expression to preserve activity of the serotonin receptor.

A method of amplified expression of a functional 5HT2c receptor, according to one embodiment (FIG. 4), may comprise: (a) preparing an MBP-5HT2c fusion protein by fusing a C-terminus of a maltose-binding protein to an N-terminus of the 5HT2c receptor without a signal sequence (step S41); and (b) expression cloning the fusion protein in a bacterial medium (step S42). The method may further comprise inserting a thrombin-cleavable linkage between the maltose-binding protein and the 5HT2c receptor. The method also may further comprise adding thrombin to separate the maltose-binding protein and the 5HT2c receptor after cloning. The method also may further comprise fusing bacterial cytoplasmic thioredoxin to residue 402 of the 5HT2c receptor.

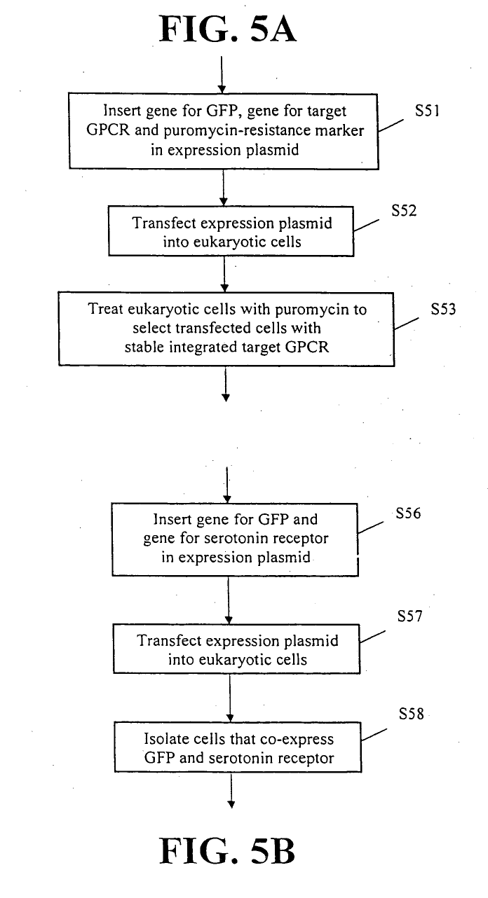

A method of amplified expression of a functional G-protein coupled receptor, according to another embodiment (FIG. 5A), comprises: (a) inserting in an expression plasmid a gene for a green fluorescent protein (GFP), a gene for a target GPCR and a puromycin-resistance marker (step S51); (b) transfecting the expression plasmid into eukaryotic cells (step S52); and (c) treating the eukaryotic cells with puromycin, after step (b), to select transfected cells with stable integrated target GPCR (step S53).

A method of amplified expression of a functional serotonin receptor, according to another embodiment (FIG. 5B), comprises: (a) inserting a gene for a green fluorescent protein (GFP) and a gene for the serotonin receptor in an expression plasmid (step S56); (b) transfecting the expression plasmid into eukaryotic cells (step S57); and (c) isolating cells that co-express the GFP and the serotonin receptor (step S58).

For example, the cells that express the serotonin receptor may be isolated by cell sorting through flow cytometry. The cells that express the GFP may be isolated by hand selection of green fluorescent colonies. According to another embodiment, a puromycin-resistance marker also is inserted in the expression plasmid, and the eukaryotic cells, after transfection of the expression plasmid into the eukaryotic cells, is treated with puromycin to select transfected cells with stable integrated serotonin receptor. The isolated cells may further be cultured and sorted to obtain additional amplification. A cytomegalovirus promoter may be applied to the expression plasmid. Binding assays may be applied to verify that the isolated cells express the functional serotonin receptor.

The method may further comprise solubilizing the serotonin receptor in a detergent system to harvest the target functional serotonin receptor. The method also may further comprise inserting additives in the expression plasmid to preserve activity of the solubilized serotonin receptor.

Effective and general expression cloning methodologies for the expression of functional GPCR proteins into biological membranes are described herein, which may be applied to the production of, for example, functional serotonin receptors. Recombinant DNA are stably integrated into, according to one embodiment, cultured mammalian cells selected for highly amplified expression of relevant receptors. Appropriate expression plasmids may also be introduced into bacterial cells for high-level expression of function receptors.

Purification and biochemical characterization methodologies are described herein which may include detergent solubilization methodologies for isolating receptors from cell membranes and chromatographic separation procedures for purifying them. The functional state of the recombinant receptor molecules may be characterized by, for example, ligand binding measurements, both on cell membranes and as purified protein. Other biochemical properties of the purified receptors may also be analyzed.

Methodologies for production of receptor complexes stabilized with protein ligands are described herein, which may be used to prepare complexes of receptor molecules with protein ligands that can be expected to stabilize the proteins into a fixed conformation and to enhance the probability for crystallization by increasing the hydrophilic surface area. Candidates include cognate heterotrimeric G proteins and antigen-binding fragments from conformation-sensitive monoclonal antibodies.

In addition, methodologies for the crystallization and structure determination of GPCR and pertinent complexes are described herein.

Analysis of signal transduction mechanisms may use the obtained structural information to develop hypotheses regarding biophysical mechanisms for GPCR signal transduction. Such hypotheses may be tested through analysis of site-directed mutant variants, complexes with relevant ligands, and cellular assays of function.

Elements and/or features of different illustrative embodiments may be combined with each other and/or substituted for each other within the scope of this disclosure and appended claims. For example, in some additional embodiments, the purification methodologies described herein, unless specifically stated otherwise, may be applied to eukaryotic cell expression as well as to bacterial expression.

### First Set of Experimental Details

Some embodiments of the subject methodologies as applied experimentally to some G-protein coupled receptor are described exemplarily below. One experiment corresponds to expression of 5HT2c in one type of mammalian cell. Other experiments correspond to expression of 5HT2c and other GPCRs in one type of bacterial cell (Escherichia coli). The methodologies described herein (and other aspects of this disclosure) may be adapted for other G-protein coupled receptors (for example, 5HT2b, 5HT1c, other serotonin subtypes, other biogenic amine receptors, such as dopamine receptors, epinephrine receptors, norepinephrine receptors, histamine receptors, and neurotensin receptors), expression in other types of eukaryotic cells, and expression in other types of bacterial cells.

A cell perceives its environment through receptor molecules embedded in the plasma membrane and endowed with selective sensitivity toward various stimuli. Conformational changes or associations that occur when a receptor interacts with an external stimulus are transmitted to the cell interior where responses are induced, often elicited through a cascade of signal transduction events. The mechanism of signal transduction depends on molecular characteristics of the receptor. There are several classes of receptors. In addition to those linked to downstream elements by heterotrimeric G proteins, there are many receptors linked to protein tyrosine kinases, ones linked to ion channels, and diverse receptors coupled in other ways as in the TGFβ/Smad, Notch and Wnt systems.

The stimulus detected by a receptor may be physical (for example, light or an electrostatic potential), but in most cases the stimulus is a chemical ligand. Some ligands are macromolecules, and others are small compounds. Some are diffusible, and others are associated with another cell or the extracellular matrix.

The most salient molecular characteristic of G-protein coupled receptors (GPCRs) is a pattern of seven hydrophobic segments that correspond to transmembrane α-helices. Therefore, GPCRs are also known as seven transmembrane (7TM) receptors. This 7TM pattern was first seen in sequences of rhodopsins (Ovchinnikov et al. 1982; Hargrave et al. 1983; Nathans et al. 1983) and a little later in the sequence of the hamster β2-adrenergic receptor (Dixon et al. 1986). The involvement of heterotrimeric G proteins in signaling through 7TM receptors was first worked out for the β2-adrenergic receptor (Ross et al.), where binding of the hormone epinephrine activates Gas which in turn stimulates adenylyl cyclase production of the second messenger cyclic AMP. The parallel role of the G protein transducin in visual signaling, where photoactivation of rhodopsin stimulates cyclic GMP phosphodiesterase and sodium-channel closure, was discovered a little later (Stryer 1986). Taken in this context, the evident homology between these two biologically disparate 7TM receptors prompted the realization that they were the founding members of the GPCR family.

A flood of GPCR clonings ensued, including the first for serotonin receptors (Julius et al. 1988; Kobilka et al. 1987) and the discovery of the huge sub-family of odorant receptors (Buck et al. 1991). A total of 948 GPCR genes were identified in a recent analysis of the human genome sequence (Takeda et al. 2002), up somewhat from the 616 found initially (Venter et al. 2000). These receptors include sensors of exogenous stimuli, such as light and odors, and others that respond to endogenous ligands ranging from cationic amines such as serotonin, to peptides such as angiotensin, and further on to proteins such as chemokines and glycoprotein hormones.

Ligand binding (retinal photoisomerization in opsins) activates a GPCR to serve as a nucleotide exchange factor for the cognate heterotrimeric G protein. Each heterotrimer is a labile association of the GTPase component, Gα, and a Gβ:Gγ heterodimer. Gα(GDP):Gβ:Gγ dissociates to Gα(GTP) and Gβ:Gγ when stimulated by an activated GPCR, and the trimer reassociates after GTP hydrolysis. Both components are tethered to the membrane, by N-terminal myristolation or palmitoylation of Gα and C-terminal prenylation of Gγ, and after activation they can diffuse away from the receptor to effector sites on their membrane-associated targets.

There are at least 15 different Gα proteins, 5 Gβs and 5 Gγs (Conklin et al. 1993), and different combinations are selective for specific GPCRs and for target effector molecules (Gilman 1987). In particular, Gαs(GTP) stimulates adenylyl cyclase, whereas Gαi(GTP) inhibits it, and Gαq(GTP) stimulates phospholipase C-β. Crystal structures have been determined for Gα proteins in various states, including a complex between Gαs(GTP) and the catalytic portion of adenylyl cyclase, of Gβ:Gγ and of heterotrimers (Noel et al. 1993; Coleman et al. 1994; Wall et al. 1995; Lambright et al. 1996; Sondek et al. 1996; Tessmer et al. 1997).

GPCR receptors are thought to exist in equilibrium between inactive and active states, which naively correspond to empty and ligand occupied receptors. The ligand-binding site is known from studies on rhodopsin (Thomas et al. 1982) and on β2-adrenergic receptor (Strader et al.) to be located between helices near the center of the membrane. Conformational changes that accompany ligand binding or activation are linked to receptor binding of the G protein. G protein association with a receptor increases its ligand affinity.

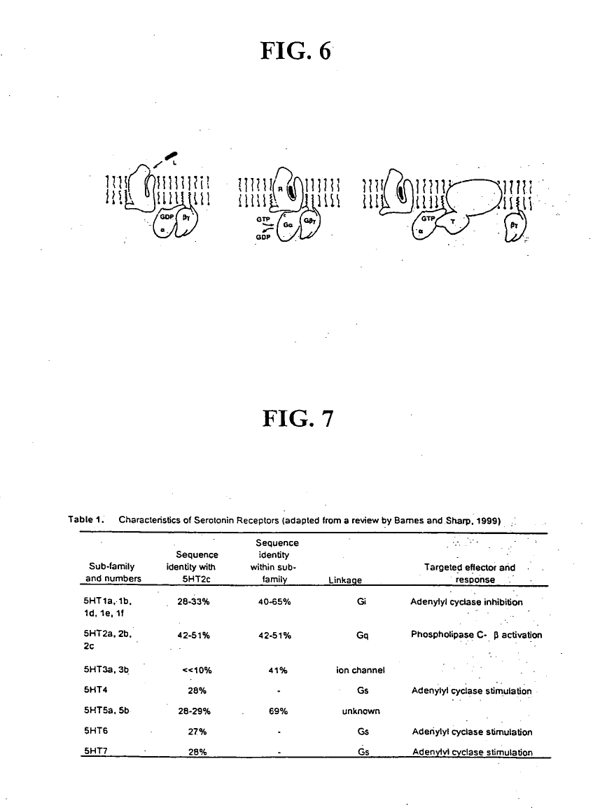

A model of G-protein coupling of GPCR activation to effector targets is exemplified in FIG. 6, which shows coupling of ligand (L) binding to a GPCR receptor (R) through catalysis of GTP for GDP exchange in Gα and dissociation of free Gα (GTP) to interact with an effector target (T). Components here are based on known structures of rhodopsin, Gα:Gβ:Gγ, and Gα(GTP):adenylylcyclase.

Many of the receptors that act in synaptic neurotransmission are GPCR family members. These include ATP and biogenic amines such as acetylcholine, dopamine, epinephrine, histamine and serotonin. Certain neuroactive peptides such as enkephalin and substance P can also act as neurotransmitters. These chemicals are synthesized in the pre-synaptic neuron, typically packaged into exocytic vesicles, and released into the synapic cleft upon neural excitation. They then diffuse across to the postsynaptic cell, typically another neuron or a muscle cell, where they can bind to cognate receptors. A specific presynaptic uptake transporter or an inactivating enzyme is usually used to remove the neurotransmitter from the site of action.

The importance of neurotransmission mediated by serotonin receptors is evident from the receptor pharmacology and tissue distribution in animal models (Barnes et al. 1999) and from pharmacological responses in humans (Kandel 2000). Serotonin receptors are a major site of action of certain mind-altering drugs, notably lysergic acid diethylamine (LSD). Depression can often be treated effectively with drugs that act on serotonergic pathways, notably by serotonin reuptake inhibitors such as fluoxetine (Prozac).

Mammals have at least fourteen structurally and pharmacologically distinct receptors for serotonin, 5-hydroxytryptamine (5HT), all but two of which are GPCR receptors. The 5HT3 exceptions are ligand-gated ion channels. The receptors are classified into seven evolutionary sub-families, members of which share common signaling linkages (Barnes et al. 1999), as shown in FIG. 7. As among many other GPCRs, the sequence similarity between members of different sub-families is low (˜30% identity), and even within a sub-family there is substantial variation (40-70% identity). Orthologs of a given receptor type, however, are highly conserved (for example, rat and human 5HT1a receptors are 89% identical).

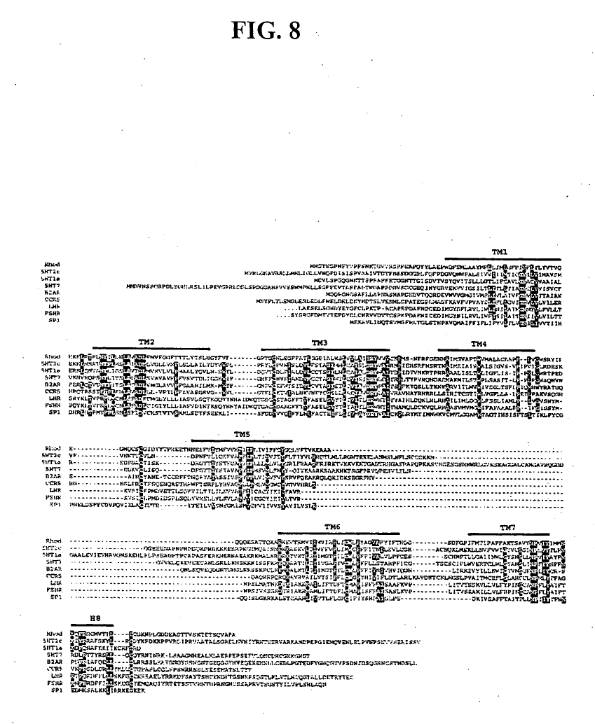

The sequences of certain 5HT receptors are compared with other GPCRs in FIG. 8, which shows structure-based sequence alignment of GPCR sequences. Sequences are from the following sources: Rhod, bovine rhodopsin; rat 5HT2c; human 5HT1a; mouse 5HT7; B2AR, human β2 adrenergic receptor; human CCR5; LHR, human leuteinizing hormone from residue 321; FSHR, human follicle stimulating hormone from residue 32; and mouse SP1 olfactory receptor. Bars over the sequences represent transmembrane helices TM1-TM8 and C-terminal helix H8, respectively, as defined in the rhodospin crystal structure. Highlighted residues designate identities or certain close similarities.

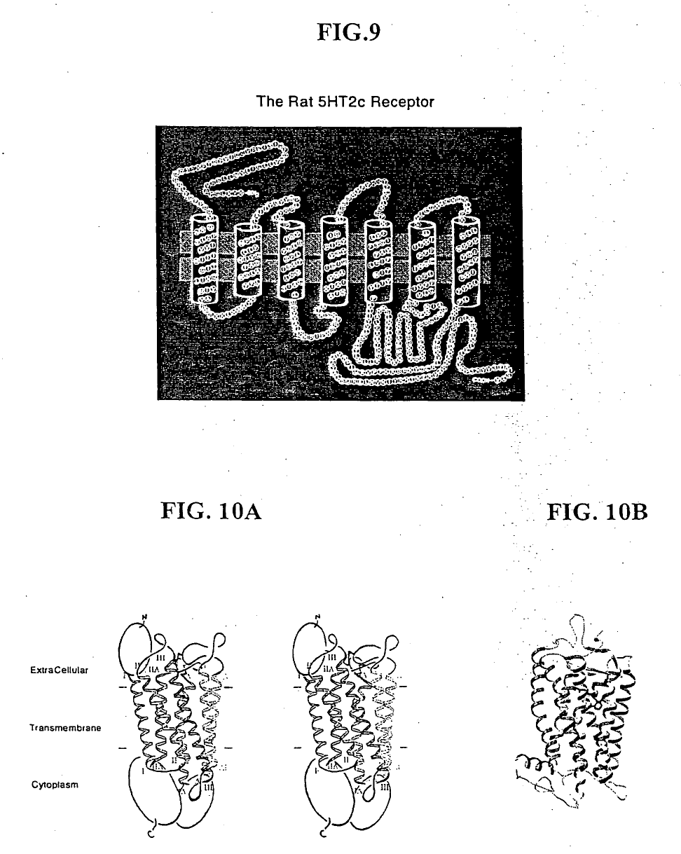

The 5HT2c receptor (then called 5HT1c) was first cloned from rat (Julius et al. 1989) and found to have a predicted size of 460 amino-acid residues corresponding to a molecular mass of 51,899 Daltons. A schematic diagram of the amino-acid sequence and assignment of transmembrane segments is shown in FIG. 9. The receptor is coupled to Gq and thereby activates phospholipase C mechanisms. The receptor triggered malignant transformation when transfected into cultured NIH 3T3 fibroblast cells. Injection of transformed foci into nude mice generated solid tumors (Julius et al. 1989). Cell lines derived from the tumors showed amplified expression of 5HT2c and were shown to bind ligands known to interact with the 5HT2c receptor, notably the agonist LSD and the antagonist mesulergine, with high affinity (K_{d}(LSD)=2.2 nM). These cells provided a starting point for structural studies on the serotonin receptor, which can exploit the rich pharmacology on 5HT2 receptors (Barnes et al. 1999), including other agonists and antagonists and also partial agonists such as lisuride and inverse agonists (mianserin and ketanserin) that compete for binding by shifting the equilibrium between active and inactive states.

The characteristic 7TM pattern of hydrophobic segments in GPCRs provides powerful constraints on possibilities for 3D structure. Moreover, this pattern when seen in rhodopsin was reminiscent of that in bacteriorhodopsin where the structure from purple membranes had shown the disposition of helices [Henderson et al. 1977]. Although the sequences showed no detectable homology and these two photoreceptors have very different biochemical actions they do both use a Schiff-base linked retinal to detect light. The topological connections in bacteriorhodopsin were found at high resolution (Henderson et al. 1990). Electron crystallography also showed that the helices in rhodoposin are disposed generally as in bacteriorhodopsin. As sequences accumulated, conserved features were realized and comprehensive alignments were made (Probst 1992). The vast majority of GPCR sequences are homologous with rhodopsin, but there also are groups of the superfamily which are atypical. These include the secretin and metabotropic glutamate receptors.

GPCR sequence alignments reveal many features besides the 7TM pattern, some of which are evident in the subset shown in FIG. 8. Most strikingly, there is substantial conservation in the transmembrane segments. There is, however, great variation in size as well as sequence for the N- and C-terminal segments and also for most interhelical loops. Some N-termini, for example, those of glycoprotein hormone receptors, include very large domains (not shown in FIG. 8). The cytoplasmic 5-6 loop is extremely variable in size. By contrast, the extracellular 2-3 and cytoplasmic 3-4 loops are relatively constant in size. The positions of several functional sites are also in common typically. These include a disulfide bridge between the N-terminus of TM3 and the 4-5 extracellular loop, N-linked glycosylation (often before TM1), a palmitoylation site at the end of H8, and phosphorylation sites in loop 5-6 and the C-terminal segment (Lefkowitz 2000).

Structural models have also been predicted from GPCR sequences (Zhang et al. 1993; Shacham et al. 2001). The best constrained model came from combining the structure of frog rhodopsin, determined at 9A resolution by electron microscopy of 2D crystals (Unger et al. 1997), with an analysis of the sequences of some 500 rhodopsin-family members (Baldwin et al. 1997). The result was an alpha-carbon template for the transmembrane helices for the rhodopsin family of GPCR receptors. Based on the Baldwin template, a rough three-dimensional model of the 5HT2C receptor was produced (FIG. 10A). Subsequently, the structure of bovine rhodopsin was reported at 2.8 Å resolution (Palczewski et al. 2000), and later refined to 2.6 Å resolution (Okada et al. 2002) (FIG. 10B). It confirms predictions based on the alpha-carbon template and adds rich detail on rhodopsin in the inactive, 11-cis retinal state.

FIGS. 10A and 10B show structural models of G-protein coupled receptors. A stereodiagram of the 5HT2c serotonin receptor is shown in FIG. 10A. The alpha-carbon template of Baldwin et al. (1997) for 7TM helices (FIG. 3C of that paper) has been elaborated with extramembranous portions drawn in rough proportion to the length or mass of these segments in 5HT2c. FIG. 10B shows a ribbon diagram of bovine rhodopsin (Okada et al., 2002) drawn in a similar orientation. FIGS. 10A and 10B are inverted from the rhodopsin convention to the cytoplasm-down orientation more commonly used for cellular receptors.

The natural abundance of rhodopsin in retinas makes this an exceptional GPCR protein. Most other GPCRs, including the serotonin receptor, are naturally scarce and therefore require the development of appropriate recombinant expression systems to support structural studies. Accordingly, methodologies for the production of recombinant serotonin receptor in sufficient yields for structural analysis and functional characterization were devised.

Integral membrane proteins present formidable, but not insurmountable problems for structural analysis. There have been striking successes starting with the first result in three dimensions, by electron crystallography at 7 Å resolution, on bacteriorhodopsin (Henderson et al. 1975) and the first atomic-level structure, at 3 Å resolution by x-ray crystallography, on a photosynthetic reaction center (Deisenhofer et al. 1985). Membrane-protein structures have been determined at an accelerated pace in recent years, and many of these new structures have had dramatic impact as in the cases of cytochrome c oxidases (Iwata et al. 1995; Tsukihara et al. 1996) and bacterial potassium and chloride channels (Doyle et al. 1998; Dutzler et al. 2002). Nevertheless, the structural output on membrane proteins is a very small fraction of that for soluble macromolecules. Through 1999, there were 12,896 PDB entries and 5348 novel macromolecular structures meeting the criteria for Macromolecular Structures (Hendrickson et al. 2000). The PDB total was nearly 19,000 in September 2002 (www.rcsb.org/pdb). Thus, while membrane proteins comprise 20-30% of all proteins in both prokaryotic and eukaryotic organisms (Wallin et al. 1998) they are but a fraction of a percent of those with known structure.

It is, of course, the natural association of membrane proteins with lipid bilayers that complicates their structural analysis. In the case of crystallography, for example, once suitably ordered crystals are obtained for a membrane protein, the diffraction analysis is as straightforward as it is for aqueous soluble macromolecules. By contrast, the biochemical preparation of pure membrane proteins is intrinsically much more challenging than for naturally soluble counterparts. One may isolate them in bilayers, either as naturally enriched or reconstituted, or make them water soluble in detergent micelles. Two-dimensional membrane arrays may be used for electron crystallography. There now are a few such atomic level structures. The membrane array also may be used for solid-state NMR experiments, and this technology is just coming of age. Soluble detergent micelles can be used for solution NMR experiments. New TROSY techniques are promising. Soluble detergent micelles also may be used for x-ray crystallography, which has dominated the field and is the approach of choice for the project proposed here. The crystallization of proteins in detergent micelles has its own special difficulties, including the following: (1) the protein may not be stable outside the lipid bilayer (Bowie 2001), (2) detergent interactions that occur during crystallization are important (Loll et al. 2001), and (3) the detergent-covered lipophilic surfaces are flexible and unavailable for lattice contacts (Ostermeier et al. 1997), which theoretically reduces the probability of crystallization by a high power of the fractional surface area (Kwong et al. 1999).

Problems that arise in the recombinant expression of membrane proteins are even more limiting than difficulties in purification and crystallization. There have been recent successes in producing recombinant bacterial proteins for analysis. Eukaryotic membrane proteins have been strikingly recalcitrant in expression at the scale needed for structural analysis. Although there are structures of important eukaryotic membrane proteins, they have all come from natural sources except for the peripheral, single-leaflet associated cyclooxygenases (Picot et al. 1994). Many mammalian membrane proteins of interest, including the serotonin receptor, are scarce and cannot be prepared from natural membranes as for retinal rhodopsin (Okada et al. 2000). Moreover, GPCR systems do not exist in prokaryotes and thus bacterial homologs, exploited so effectively in potassium channel studies (Doyle et al. 1998), are not an option in this case. Appropriate recombinant expression systems are therefore needed to support such structural studies. Various expression systems are being adapted for the production of mammalian membrane proteins (Grisshammer et al. 1995).

A study of the structural biology of G-protein coupled receptors (GPCRs) may be multi-faceted. One dimension may include the study of, for example, the neurobiology of olfaction and neurotransmission. It has been found, for example, that the perception of smell begins with a large class of specific GPCRs, the olfactory receptors (Buck et al. 1991). Another facet may include studying structural aspects of signal transduction, such as analyzing structures of several molecules involved in signaling through protein tyrosine kinases, which may include the following: protein ligands such as fibroblast growth factor (FGF) (DiGabriele et al. 1998), stem cell factor (Jiang et al. 2000), and ciliary neurotrophic factor (CNTF) (McDonald et al. 1995); tyrosine-kinase portions from the insulin receptor (Hubbard et al. 1994) and lymphocyte kinase (Yamaguchi et al. 1996); and ligand-binding portions from an FGF receptor (Stauber 2000) and the angiogenesis receptor Tie-2. In addition, other structural studies have been conducted on CD4 (Ryu et al. 1990; Ryu et al. 1994; Wu et al. 1997; Wu et al. 1996) and CD8 (Leahy et al. 1992), which are tyrosine-kinase linked components of the cellular immune response. Signaling through the histidine-kinase receptors of two-component systems has also been studied, and the structure of the histidine-kinase portion of PhoQ (Marina et al. 2001) and a number of the sensor domains from such receptors were determined.

Serotonin receptor 5HT2c is a suitable entry point for structural efforts on GPCRs for the following reasons: (1) there is a body of work on the neurobiology of this and related serotonin receptors, (2) a mouse fibroblast cell line which is available has been shown to express the receptor at a high level (Julius et al. 1989), and (3) there are well characterized ligands for this receptor. Methodologies for amplified expression in mammalian cells and in bacterial cells as a fusion protein are described herein. Recombinant receptors expressed in either membrane system are shown to have wild-type activity. Additional methodologies are described herein which may be used to solubilize, purify and crystallize the receptor molecules from both systems. Methodologies established in the 5HT2c studies have been adapted for the expression of 5HT1a, 5HT1b and 5HT7. Structural studies on other GPCRs, including olfactory receptor, glycoprotein hormone receptors, and chemokine receptors used by HIV, have also been undertaken and are discussed below.

Amplified Expression of Functional 5HT2c in Mammalian Cells

Expression of the rat 5HT2c serotonin receptor in the initial mouse fibroblast cell line, NIH 3T3, was at a level of 10³ to 10⁴ high-affinity binding sites for ¹²⁵I-labelled LSD per cell (Julius et al. 1988). The subsequent tumor-cell lines showed enhanced expressions levels of up to 8×10⁵ receptors per cell (Julius et al. 1989), which compares favorably to the estimated natural level of approximately 10⁵ receptors per cell of the choroid plexus. Production of mammalian proteins for crystallography in Chinese hampster ovary (CHO) cells was successful in connection with extracellular portions of the T-cell coreceptors CD4 and CD8. An expression plasmid coupled to dihydrofolate reductase (DHFR) and driven by the cytomegalovirus (CMV) promoter was devised to produce CD8 (Leahy et al. 1992). Upon transfection into DHFR-deficient CHO cells, those cells exhibiting multi-copy integration at optimal sites are expected to show reduced susceptibility to the DHFR inhibitor, methotrexate. Selection for methotrexate resistance was then used to amplify CD8 expression. Attempts were made to improve upon 5HT2c expression in 3T3 cells. In one approach, tumor-derived mouse fibroblast cell lines expressing the 5HT2c receptor were generated using the protocol established by Julius et al. Through multiple rounds of flow cytometry, cells were selected on their ability to respond to progressively decreasing concentrations of agonist. Cells were permeabilized with a calcium sensitive dye and the cellular response was monitored by fluorescent detection of the calcium released from the intracellular stores. Expression levels were monitored at each cycle of sorting and selection. Expression levels gradually increased to approximately 1×10⁶ molecules/cells.

In a second approach, a nine-residue epitope specifically recognized by an anti-hemagglutinin (HA) monoclonal antibody was fused to the N-terminus of the 5HT2c receptor. It was hoped to select high-expressing cells by labeling the cells with the anti-HA antibody which could be monitored in flow cytometry with a fluorescent secondary antibody. Fluorescent intensity of a given cell was expected to correlate with the amount of receptor expressed on its surface. Unfortunately any modification to the N-terminus of the 5HT2c receptor abolished expression.

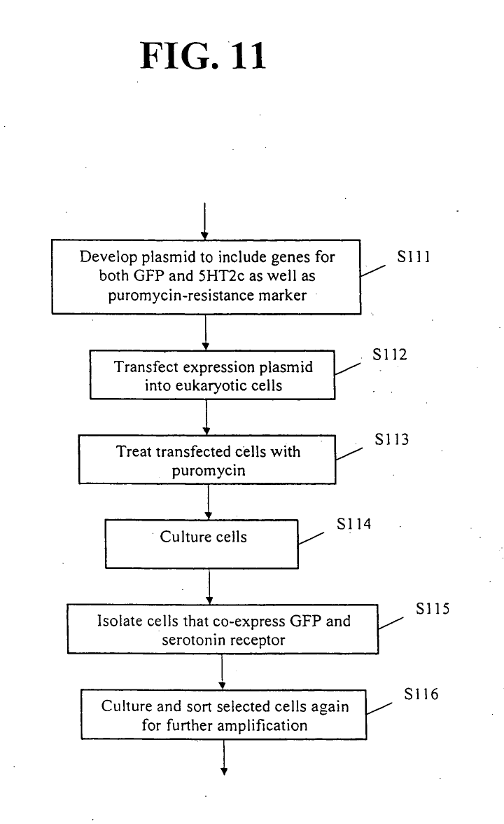

Greater success was obtained with expression in 293 human embryonic kidney (HEK) cell lines. The amplification system devised for expression of the serotonin receptor in HEK293 cells employs green fluorescent protein (GFP) as a selectable marker (FIG. 11).

GFP can be used as a selectable marker for expression in mammalian cells. GFP offers two main advantages: the intensity of fluorescence correlates with the expression levels of the foreign gene, and the highest expressing cells can be selected by FACS sorting and amplified. The expression level of a transfected and selected cell line is stable over time. Expression of GFP is correlated to the expression of the gene of interest. However, since translation is independent for GFP and the target protein, fluorescence of the former does not assure proper targeting and folding of the latter in the cell membrane. An antibody epitope may be genetically fused to the N-terminus of different serotonin receptors that are unlikely to possess a signal sequence. This tag can be used to assess expression. Once stable integrants have been selected by antibiotic resistance, a flow cytometry experiment similar to that performed with GFP can be devised. Antibodies can be fluorescently labeled and can be used for selection and amplification of transfected cells. The advantage of this approach is that fluorescence correlates directly to the number of antigen sites (and therefore receptors) on the cell surface.

A plasmid was developed to include genes for both GFP and 5HT2c as well as a puromycin-resistance marker (step S111). The puromycin-resistance marker is under control of its own promoter, and the 5HT2c and GFP genes, separated by an internal ribosome entry site (IRES), are under the control of the same strong CMV promoter used previously. Stable integrants were selected by treating transfected cells with puromycin (steps S112 and S113). Cells that had productively and repeatedly integrated GFP typically also produce the receptor in similarly high abundance (step S114). Cells that express GFP highly can be isolated readily either by hand picking of green fluorescent colonies or by cell sorting via flow cytometry (step S115). Selected cells can then be cultured and sorted again for further amplification (step S116).

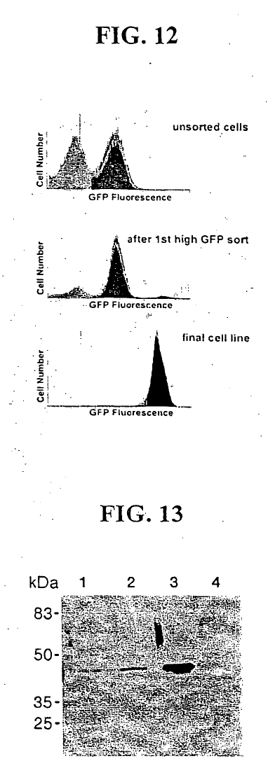

The results of GFP selection are shown in FIGS. 12 and 13. FIG. 12 shows flow cytometry sorting of GFP-serotonin receptor expressing HEK 293 cells. The populations are represented progressively darker in accordance with increasing levels of GFP expression. FIG. 13 shows western blot analyisis of cells at different stages of selection. Lane 1 represents cells 48 hours after transfection. Lane 2 represents cells after puromycin selection. Lane 3 represents cells after GFP selection. Lane 4 are untransfected cells. Twenty thousand cells were run on each lane, and the samples were deglycosylated for one hour on ice with endoglycosidase F prior to loading. The membrane was probed with an anti-5HT2c polyclonal antibody (Backstrom 1995).

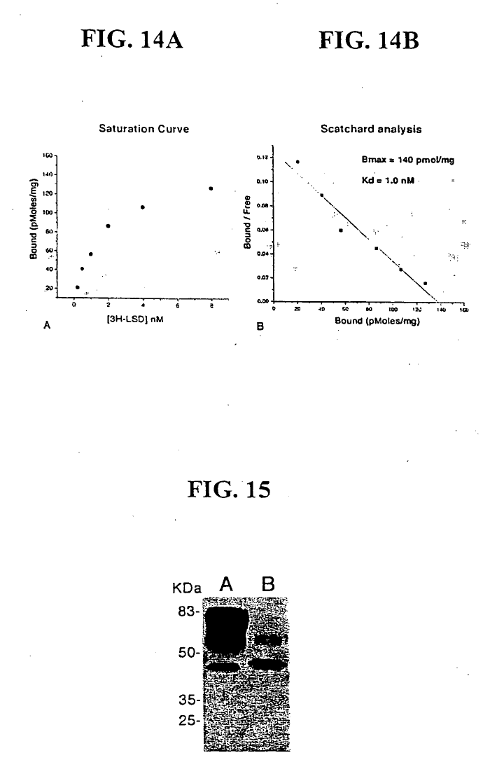

Binding assays were performed in order to verify that the selected cell cultures did express functional serotonin receptors and to quantify expression levels. Tritiated LSD was bound to membranes isolated from enriched cells through saturation (FIG. 14A) and a Scatchard analysis of these data was made to quantify the binding (FIG. 14B).

The data show that the ligand bound with Kd ˜1.0 nM (as seen in vivo) and that these membranes expressed ˜140 pmoles of functional receptor per mg of membrane protein. On a per cell basis, this corresponds to ˜1.5×10⁶ LSD-binding sites per cell, and these cell lines do not diminish over time. This level of expression is sufficient, at least in principle, to support structural studies. Four ten-layer cell farm runs suffice to produce ˜500 mg of total membrane protein which corresponds to 3.5 mg of functional receptor in the cell membrane.

Tags of various nature suitable for purification of the receptor were genetically fused to its C-terminus without hindering expression levels or activity. Once effective expression protocols were established and the products could be purified, characterized and set up for crystallization (see discussion below), attempts were made to remove potential sources of conformational heterogeneity that might interfere with crystallization. The glycosylation pattern of the 5HT2c receptor expressed in mammalian cells was analyzed. Western blot analysis of cells harvested from a stable 293 cell line expressing the 5HT2c receptor was performed.

FIG. 15 shows a western blot probed with anti-5HT2c antibody in which cells of stable 293 cell line expressing the 5HT2c receptor were run on an SDS-PAGE gel, without and with the addition of a deglycosylation enzyme. Lane A represents approximately 20,000 cells. Lane B represents the same number of cells after treatment with endoglycosidase F. The membrane was probed with anti-5HT2c rabbit polyclonal antibody. This is as expected from the 5HT2c sequence (FIG. 8), which has three extracellular NxS/T sites for potential N-linked glycosylation at positions 39 (N terminus), 204 and 205 (4-5 loop).

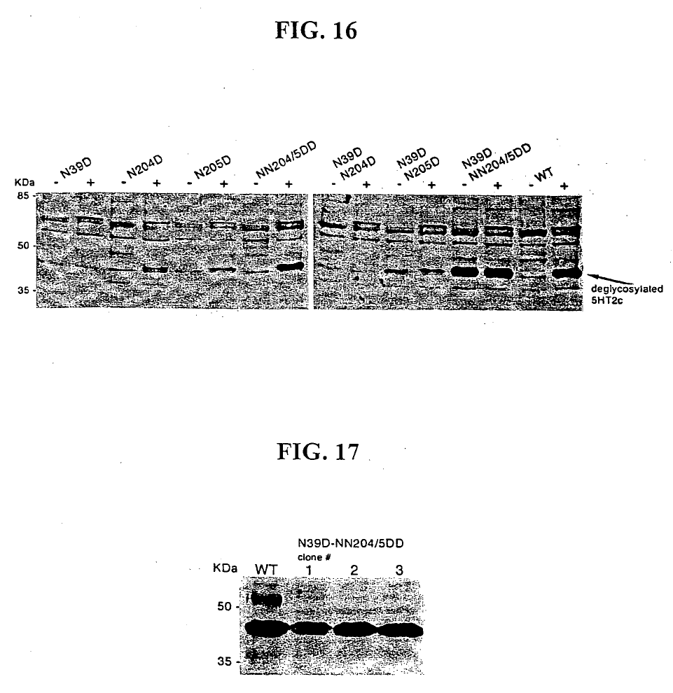

Each asparagine residue at a potential glycosylation site on 5HT2c was mutated to aspartic acid. Constructs were generated to express each single mutant individually (N39D, N204D and N205D), as the three possible combinations of double mutants, and as the triple mutant. These constructs, together with the expression plasmid encoding the wild type receptor were transfected into 293 cells. The cells were harvested 48 hours after transfection. Each population of cells was divided into two, one of which was deglycosylated by treatment with endoglycosidase F. Products were analyzed by western blot (FIG. 16).

FIG. 16 shows a western blot analysis of cells transfected with glycosylation-site mutants of the 5HT2c receptor. The mutant and wild-type receptors were each run in two lanes. The identity of the mutation(s) is shown above the corresponding lanes. Wild-type receptor, run in the last two lanes, is labeled ‘WT’. The alternating ‘−’ and ‘+’ signs correspond respectively to cells that were untreated and cells subjected to deglycosylation. The membrane was probed with an anti-5HT2c rabbit polyclonal antibody.

Surprisingly, expression levels of the 5HT2c receptor varied considerably amongst the different constructs. The single and to some extent also the double mutants seemed to express less than the triple mutant. The expression level of the triple mutant appeared comparable to that of wild type receptor. Overall expression levels were low, making it possible to detect only the non-glycosylated or deglycosylated form of the receptor. Nevertheless, one can conclude from this experiment that all three potential sites are indeed glycosylated. One can also conclude that a non-glycosylated form of the receptor can be expressed at levels comparable to that of the wild-type protein.

A stable cell line expressing the triple-mutant form of the receptor which cannot be glycosylated was generated using GFP as a marker. The three most fluorescent clones that could be selected by flow cytometry were amplified and tested for expression levels.

FIG. 17 shows a western blot analysis of individual clones generated from cells expressing 5HT2c receptor mutated at three sites, N39D, N204D and N205D. The three clones are numbered 1 through 3 above the corresponding lanes. ‘WT’ refers to cells expressing the wild type receptor. The same number of cells was run on each lane. Only the cells expressing the wild type receptor were deglycosylated before running on the gel. FIG. 17 shows that each clone expresses the receptor at levels comparable to those of a cell line expressing the unmodified, wild type receptor. This mutant form of the receptor was shown, by performing activity assays, to have a ligand-binding profile indistinguishable from that of the wild-type protein.

Cell lines were also generated in which the conserved, and most probably palmitoylated, cysteine after the seventh transmembrane domain (C387) was mutated to either alanine or serine. Neither the expression levels nor ligand-binding profiles were affected in these mutant receptors.

Expression of Functional 5HT2c as Fusion Protein in E. coli

There are advantages and disadvantages to alternative expression systems. The expression of a mammalian receptor in mammalian cells offers the advantage of presenting the expressed gene with the proper translational and membrane-insertion machinery for functional translocation to the cell surface. On the other hand, mammalian derived cell lines require great expense and technical expertise. In addition, the generation of highly expressing cell lines is troublesome and time consuming. This limits the number of mutant variants that can be examined in a reasonable amount of time. Prokaryotic expression has great advantages. Bacteria are relatively simply to grow, easy to scale, and most importantly, the time between genetic construction of a target and its expression as protein is short. Moreover, structural analyses might benefit from having molecules that lack post-translational modifications such as glycosylation and palmitoylation, which can negatively affect the formation of highly ordered crystals. This potential advantage can be a detrimental disadvantage if such post-translational modifications are required for correct folding and function of the protein to be expressed. Work on the 5HT2c receptor expressed in mammalian cells showed that neither glycosylation nor palmitoylation are essential requisites for this receptor. The major caveat with bacterial expression of eukaryotic membrane proteins is whether receptors can indeed be functionally inserted into the plasma membrane of a bacterial cell.

Initial attempts to express functional 5HT2c receptor in E. coli centered on the observation that the processed N-terminus of the receptor expressed in 293 cells actually begins at Ile33 rather than at the start of translation. This datum was shown by N-terminal sequencing of purified protein expressed in 293 cells. The presence of a signal sequence in the 5HT2c receptor had been hypothesized but never unambiguously demonstrated (Abramowski et al. 1995). It was thought that expression could be achieved by substituting the endogenous, previously unidentified signal sequence, with one of bacterial origin. Several different bacterial signal sequences were tried, including the signal sequences for the outer membrane protein gene ompT, and for the periplasmic and inner membrane protein genes malE, lacY and pelB. These experiments resulted in only low levels of expression, although some specific activity was observed. No specific activity was detected when the receptor was expressed with its endogenous signal sequence nor in the absence of any signal sequence.

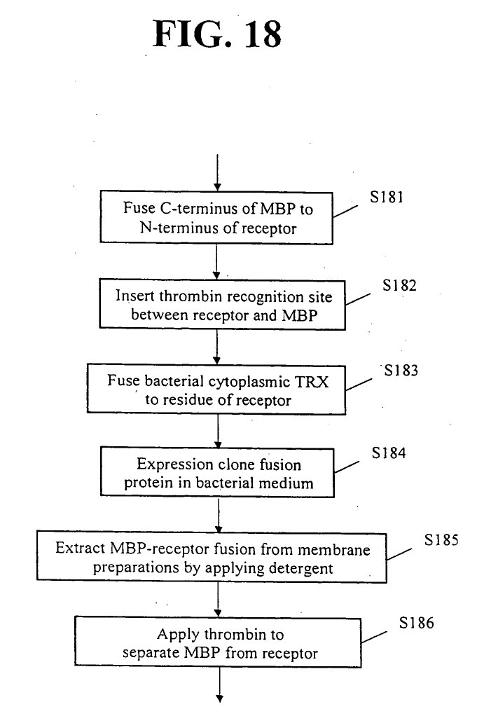

Next, another method of amplified expression of a functional G-protein coupled receptor was developed (FIG. 18). The N-terminus of the receptor lacking its signal sequence was fused to the C-terminus of maltose-binding protein (MBP), a protein that is secreted into the bacterial periplasm (step S181).

A rationale for this methodology is that the translation and membrane translocation machinery of the bacterial cell is ‘primed’ by the bacterial leader protein and that the heterologous receptor protein follows in course to be inserted into the membrane. An expression system for the neurotensin receptor has been described (Grisshammer et al. 1993). Functional expression of a GPCR as a fusion to MBP has been shown to work for the β-adrenergic receptor (Hampe et al. 2000), the A2 adenosine receptor (Weiss et al. 2002), and the M2 muscarinic receptor (Furukawa et al. 2000).

Extensive optimizations of the expression conditions were performed. The variables screened included the bacterial strain, the media used for bacterial growth, the strength of the promoter, the IPTG inducer concentrations and the temperature of incubation.

Several secretion vectors were engineered for ready interchange of receptor constructs. Maltose-binding protein (MBP) fusions were used in each of these, but various other signal sequences were tried. Fusions were constructed with thrombin-cleavable linkages of various lengths; and several short, non-cleavable linkages were also made. Based on N-terminal sequencing of receptor purified from HEK 293 expression of 5HT2c, the processed N-terminus actually begins at Ile33 rather than at the start of translation. The MBP side of the shortened junctions was defined by the degree of ordering (B factors) in the crystal structure, which ends in a C-terminal helix.

The choice of the construct appeared to affect expression levels considerably. The length of the linker between MBP and the receptor was varied, and a recognition site for thrombin was inserted (step S182). Short linkers seemed to express better than long ones.

Surprisingly the C-terminal fusion of the bacterial cytoplasmic protein thioredoxin (TRX) to residue 402 of the receptor (15 residues after the palmitoylated cysteine 387) increased expression levels substantially (step S183). This amplification does not occur if TRX is fused to the C-terminus of the receptor, although TRX fused to the C-terminus of the neurotensin receptor has been shown to promote an increase in expression levels (Tucker et al. 1996). Ultimately, the expression was increased from an initial yield of ˜30 μg per liter of culture to between 500 μg and 1 mg per liter.

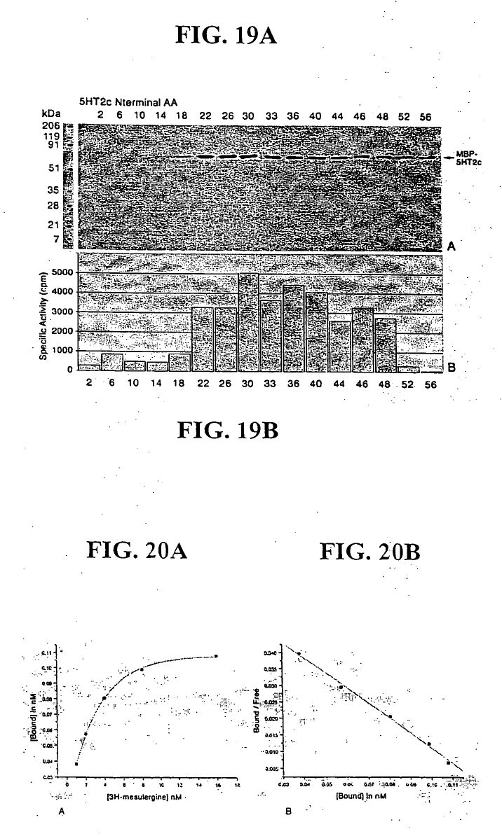

Different constructs and different variables that were screened were compared for resulting expression by running a fixed amount of cells on a gel and performing western blot analysis. Specific relative activity was assayed by converting an equal number of cells to spheroplasts and screening at a fixed concentration of radioligand. Data from a typical experiment with expression of various MBP-5HT2c fusion constructs is shown in FIGS. 19A and 19B. MBP was fused to 5HT2c at different positions spanning from the unprocessed N-terminus to the likely start of the first transmembrane region. FIG. 19A shows a western blot probed with anti 5HT2c antibody. The numbers above each lane refer to the first residue of the 5HT2c receptor fused to MBP. An equal number of cells were loaded in each lane. FIG. 19B shows relative specific activity for each construct. The numbers below each lane refer to the first amino acid of the 5HT2c receptor fused to MBP. An equal number of spheroplasts were assayed for each construct. The radioligand ³H-LSD was used at 1 nM concentration. The specific activity was calculated as the difference between the total activity and the activity assayed in the presence of 10 μM mesulergine. Assays were performed in triplicate and what is shown is the average.

Once conditions were established for expression at a high level, the specific activity of the expressed protein was fully analyzed on an absolute scale, as for material from mammalian cells. In this case, experiments were carried out on spheroplasts (bacterial cells stripped of their outer membranes) using tritiated mesulergine (FIGS. 20A and 20B). FIGS. 20A and 20B correspond to data obtained from ligand binding to bacterial spheroplasts isolated from E. coli cells expressing the MBP-serotonin receptor fusion protein. FIG. 20A shows a saturation curve for tritiated mesulergine. FIG. 20B shows a Scatchard plot of the data shown in FIG. 20A. The specific activity was calculated as the difference between the total activity and the activity assayed in the presence of 10 μM serotonin. Assays were performed in triplicate and what is shown is the average of these measurements. The antagonist was found to bind with K_{d} ˜2.2 nM, which is the same as is found physiologically, and for this preparation there were 133 μg of active receptor per liter of bacterial culture.

Expression tests in E. coli can be performed rapidly since the rate-limiting step is construction of the expression plasmid. Expression cassettes were engineered in such a way that a gene can be inserted into any of several expression vectors with a single cloning step (step S184). The MBP-receptor fusion may be extracted from membrane preparations by applying the one or more detergents (step S185), as discussed below. With the thrombin recognition sequence inserted between the receptor and the MBP, the MBP can be removed quantitatively by adding thrombin (step S186).

Expression of 5HT1a, 5HT1b and 5HT7 as Fusion Proteins

Expression of three other serotonin receptors (human 5HT1A, murine 5HT1B and murine 5HT7) was investigated by genetically engineering fusions of these proteins to MBP. Different constructs were generated for each protein, and the site of fusion to MBP was varied along the N-terminus of the receptor. Some fusions were made close to the N-terminus, some close to the first transmembrane segment, and others in between the two. Experience derived from work on the 5HT2c receptor was used as a guideline in designing these constructs.

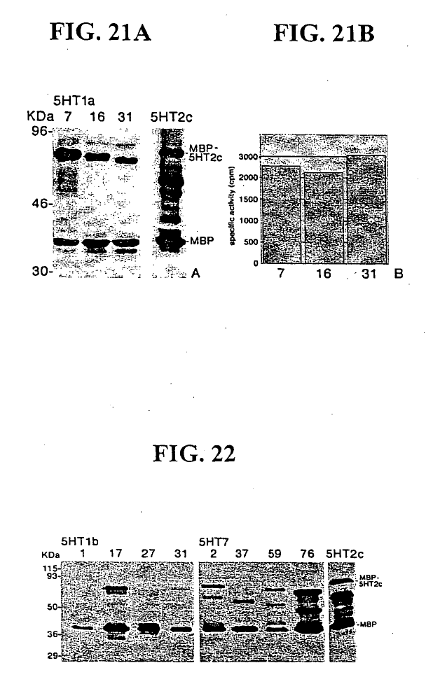

Expression data and relative activity data for the 5HT1a receptor are shown in FIGS. 21A and 21B. FIG. 21A shows a western blot probed with anti-MBP polyclonal antibody (New England Biolabs). The first three lanes, marked 7, 16 and 31 show expression of MBP fused to 5HT1a at positions 7, 16 and 31, respectively. The last lane, marked 5HT2c, shows expression of the MBP-5HT2c receptor fusion. FIG. 21B shows ligand binding assays performed on spheroplasts. ³H-5HT was used as radioligand at 10 nM concentration and the background activity was measured in the presence of 10 mM 8-OH-DPAT. Assays were performed in triplicate and what is shown is the average of these measurements. In addition, cell pellets expressing this receptor were isolated in the membrane fraction of the bacteria and extracted from these quantitatively with the non-ionic detergent dodecyl-β-maltoside. The data strongly suggest that the 5HT1a is inserted in the bacterial inner membrane.

FIG. 22 shows expression data for the 5HT1b and 5HT7. The western blots shown in FIG. 22 were probed with anti-MBP polyclonal antibody (New England Biolabs, Inc.). The first four lanes, marked 1, 17, 27 and 31, show expression of MBP fused to 5HT1b at positions 1, 17, 27 and 31. The second four lanes, marked 2, 37, 59 and 76, show expression of MBP fused to 5HT7 at equivalent positions of its sequence. The last lane, marked 5HT2c, shows expression of the MBP-5HT2c receptor fusion. Equal numbers of cells were loaded in each lane. The site of attachment of the receptor to MBP appears to be an important factor for expression.

Preparation of 5HT2c Receptors from Mammalian Cells

HEK 293 cells expressing the 5HT2c receptor were harvested and lysed osmotically. The nuclear fraction together with unlysed cells and cellular debris were pelleted by mild centrifugation (<1000×g). Crude membranes were then harvested by ultracentrifugation. Over 50 different detergents were screened and tested for their ability to extract the 5HT2c receptor in a functional form from the isolated membranes. Typically, the experiments were performed as follows.

Membranes were resuspended at a concentration of membrane protein of approximately 10 mg/ml. Radioligand was added. The membranes were then diluted two fold with buffer (control), and with different detergents at 2% minimum concentration. A higher concentration was used for those detergents that have a critical micellar concentration (CMC) of above 2%. After one hour incubation at 4° C., the detergent insoluble fraction was separated from the soluble fraction by ultracentrifugation at 100,000×g for one hour. The extraction efficiency of a given detergent was tested by quantitative western blot analysis. The activity of the solubilized fraction was assayed using small size exclusion columns to separate the bound from the free radioligand and compared to activity measurements performed on non-detergent treated membranes.

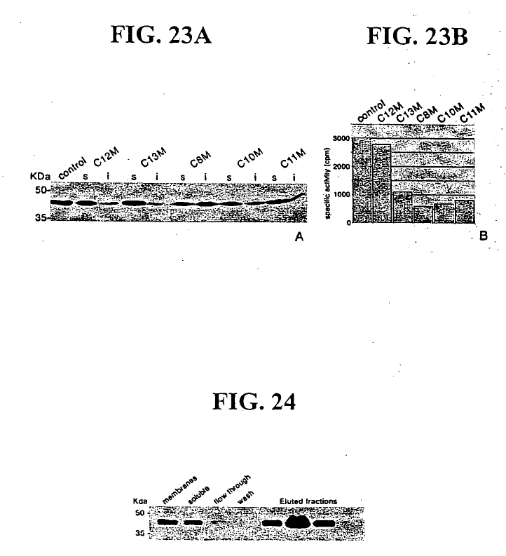

Results from such an experiment, in this case testing different maltoside detergents, are shown in FIGS. 23A and 23B, which show yield and activity of 5HT2c receptor solubilized by maltoside detergents. FIG. 23A shows a western blot probed with anti-5HT2c antibody. Control refers to unsolubilized membranes. “s” and “i” refer to soluble and insoluble fractions, respectively. The detergents used for every soluble/insoluble comparison are identified by acyl-chain length over corresponding lanes. Equal volume of sample was loaded in each lane. FIG. 23B shows a specific activity measured at 10 nM ³H-LSD. For the detergent treated samples only the soluble fraction was assayed. C12M is dodecyl-β-maltoside. C13M is tridecyl-β-maltoside. C8M is octyl-β-maltoside. C10M is decyl-β-maltoside. C11M is undecyl-β-maltoside. Parameters such as the ratio of the concentration of detergent to the concentration of membrane protein, ionic strength and pH were tested. Dodecyl-β-maltoside, FOS-choline 12 and digitonin were amongst the detergents selected for their ability to extract the 5HT2c receptor in a native-like conformation.

A deca-histidine tag was genetically engineered to the C-terminus of the receptor for purification. This tag was fused to the wild type receptor as well as to the mutant form of the receptor that cannot be glycosylated or palmitoylated (discussed above). Cell membranes were washed at both low and high ionic strength to eliminate peripherally attached membrane proteins and other contaminants. The solubilized receptor was purified by metal-affinity chromatography. Digitonin and FOS-choline 12 were the detergents used in this experiment. The purified protein could be concentrated to approximately 3-4 mg/ml.

FIG. 24 corresponds to affinity purification of 5HT2c receptor. FIG. 24 shows a western blot performed on fractions collected at various stages of purification. The western blot was probed with anti-5HT2c antibody. Labels are as follow: ‘membranes’, the initial membranes; ‘soluble’, soluble fraction; ‘flow through’, fraction that does not bind to the metal-affinity resin; ‘wash’, fraction collected from washing the metal-affinity column with a buffer containing detergent (whereby digitonin was exchanged to FOS-choline12), 250 mM KCl and 40 mM imidazole; ‘elute’, peak fractions collected by eluting the sample with buffer containing detergent, 150 mM NaCl and 400 mM imidazole.

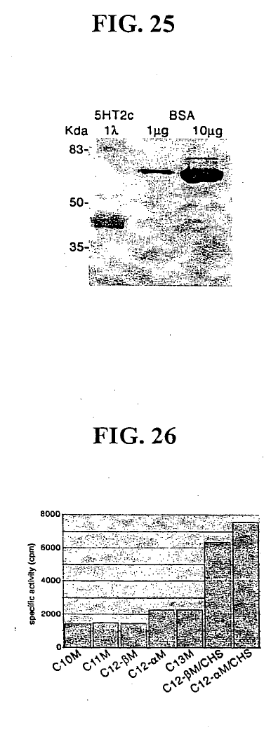

Analysis of the purified 5HT2c receptor is shown in FIG. 25, which shows a Coomassie-stained SDS-PAGE gel of purified material from the same preparation. The SDS-PAGE gel of purified material is stained with Coomassie blue to detect all protein. The first lane after the molecular weight markers was loaded with 1λ of purified material 1 and 10 μg of bovine serum albumin (BSA) were loaded in the other two lanes to serve as mass markers.

The ability of the purified material to bind ligand was assayed by radioligand-soluble binding assays. Traces of radioligand were also added to the membranes and fractions at different stages of the purification assayed for activity. Gel filtration experiments were also performed with traces of radioligand. Other purification schemes were also investigated, including ion-exchange chromatography and immunoaffinity chromatography based on an antibody epitope fused to the receptor C-terminus.

Preparation of 5HT2c Receptors from Bacterial Cells

The MBP-5HT2c receptor fusion was extracted from membrane preparations by screening the solubilization efficacy of various detergents. Following similar protocols to those for mammalian membranes, efficiency of solubilization was monitored by western blot analysis on denaturing gels, and activity was tested by soluble ligand binding assays. It was noted that the set of detergents suitable for the receptor varied between mammalian and bacterial membranes, presumably because of differences in membrane composition. In general, the bacterially-expressed receptor was found to be less stable than the one expressed in mammalian cells, again probably because of differences in lipidic composition in the two membranes. In order to stabilize the 5HT2c receptor produced in E. coli, additives were screened for their ability to preserve activity of the solubilized species. The most striking example found was cholesteryl hemisuccinate (CHS). CHS has been used successfully as a detergent additive for the solubilization and purification of other GPCRs (Weiss et al. 2002; Furukawa et al. 2000; Mirzabekov et al. 1999; Grisshammer et al. 1999).

FIG. 26 shows a comparison of the activity of the 5HT2c receptor solubilized in different detergents. In this experiment, where maltoside detergents were screened, the two isomers of dodecyl-maltoside were also compared, individually as well as with the addition of CHS. Soluble ligand-binding was assayed with the radioligand ³H-LSD at 12.5 nM concentration. CLOM is decyl-β-maltoside. C11M is undecyl-β-maltoside. C12-βM is dodecyl-β-maltoside, C12-αM is dodecyl-α-maltoside. C13M is tridecyl-β-maltoside. CHS was added to dodecyl-β-maltoside and dodecyl-α-maltoside in a 1:5 (w/w) ratio. The background activity was assayed in the presence of 10 μM mesulergine. Assays were performed in triplicate and the average of measurements is shown.

A thrombin recognition sequence was engineered between MBP and the 5HT2c receptor. MBP could be removed quantitatively by the addition of thrombin. The cleaved receptor (i.e. lacking the N-terminal MBP) was shown to bind ligand as effectively as does the uncleaved fusion protein.

Three constructs were also generated lacking a linker between MBP and 5HT2c. MBP, as shown by its crystal structure (Spurlino et al. 1991), terminates in an alpha helix. In these three uncleavable constructs, the receptor was fused to each of the last three structurally ordered residues of the C-terminal helix of MBP. The objective was to sample different MBP-receptor relative orientations in order to facilitate crystallization. These constructs expressed as well and were as active as those carrying a linker, and they were resistant to thrombin proteolysis. Cleavage of the endogenous MBP signal peptide was shown to occur by N-terminal sequencing of the purified material.

The MBP portion of the fusion was shown to be functional by binding of the solubilized complex to amylose resin. However, amylose affinity-chromatography could not be used for purification in the presence of maltoside detergents since the carbohydrate moiety of these detergents interferes with binding of MBP to the resin.

A deca-histidine tag was genetically fused to the C-terminus of the receptor preceded by a thrombin cleavage site, and purification was achieved by metal affinity chromatography. Substantial purification was also achieved prior to solubilization by isolating the bacterial membranes to remove cytosolic proteins. These membranes were subsequently washed with a sub-solubilizing concentration of detergent to eliminate many peripherally attached membrane proteins.

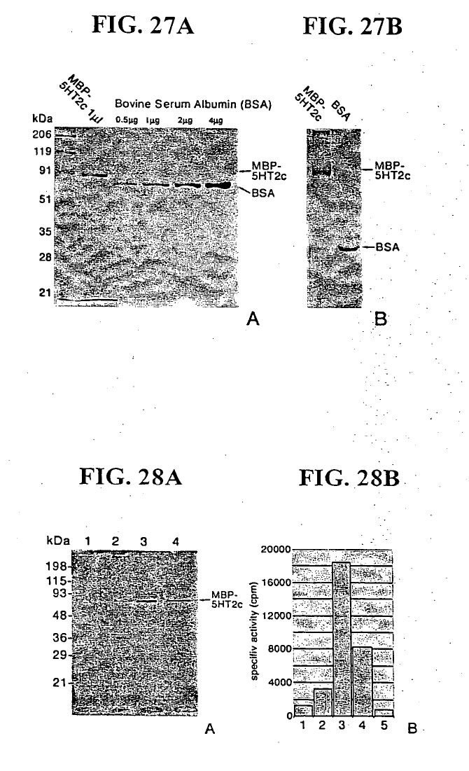

FIGS. 27A and 27B correspond to gel electrophoresis of purified MBP-receptor fusion protein, with a denaturing and a native gel of the MBP-5HT2c fusion protein, respectively. Each gel is stained by Coomassie blue. FIG. 27A corresponds to denaturing polyacrylamide-SDS gel, and 1λ of sample was loaded and compared with molecular mass standards and bovine serum albumin (BSA) concentration standards. FIG. 27B shows native polyacrylamide gel compared with BSA. Dodecyl-β-maltoside/CHS was used as detergent for the purification and for the preparation of the native gel. Each gel shows a single, sharp band indicative of homogeneity and purity.

The sample, after metal affinity chromatography, was characterized further in a variety of ways including activity assays and mass spectrometry. Mass spectrometry confirmed that the C-terminally fused deca-histidine tag could be proteolytically removed. Gel filtration was also used to improve purity and to characterize the oligomeric state of the protein. The protein was found to migrate as a broad 300 kDa and as a sharp 150 kDa protein. Both fractions were stable and bound ligand specifically as could be assayed by the gel-filtration profile of each fraction purified in a first run and run individually in a second run.

FIGS. 28A and 28B show a silver-stained denaturing gel of fractions collected from the rerun of the 150 kDa species, and the corresponding activity profile, respectively. FIG. 28A shows silver stained denaturing gel of MBP-5HT2c. The four peak fractions from a gel-filtration run were loaded sequentially in lanes marked 1 through 4. A fifth fraction, marked 5 in FIG. 28B was omitted as it did not contain protein. FIG. 28B shows activity profile of peak fractions. The sample was incubated with 1 nM ³H-LSD prior to loading on the column. Background activity was assayed by addition of 10 μM mesulergine together with the radioligand. The column was a Superose 6 HR10/30 (Pharmacia), equilibrated in 50 mM Tris/HCl pH 7.4, 150 mM NaCl, 0.15% dodecyl-α-maltoside/CHS. 1 ml fractions were collected.

The purification protocols rely on affinity tags engineered to the C-terminus of the 5HT2c receptor. A deca-histidine tag preceded by a thrombin recognition site has proven to be the most successful. C-terminal modifications do however pose some concerns. The C-termini of GPCRs are extremely diverse in both amino acid composition and length. A tag might be accessible to its target affinity matrix for one receptor and not for another. The presence of a C-terminal tag might provoke unpredictable instability to a fusion partner. Expression levels for the bacterially expressed neurotensin receptor have been shown to vary according to the nature of C-terminally-fused affinity tag (Tucker et al.). In addition, proteases (such as thrombin) cleave after their substrate recognition sequence, leaving behind several typically unstructured residues which remain on the N-terminal side, and therefore on the protein, after proteolysis. These additional residues could have a detrimental effect in a crystallization experiment.

The purification scheme preferably allows for efficient purification of any receptor independent of its identity. Affinity chromatography of the bacterial fusion protein by binding of MBP to an amylose resin has been used to great success. The genetic engineering of affinity tags in the link region between MBP and the receptor, on the MBP side of the protease site (so that the tag does not remain attached to the protein after proteolysis) may be further investigated. Additional experiments may focus on polyhistidine tags and streptagII (Shinzawa-Itoh 1995), because of the difficulties often encountered in eluting an antigen-tagged protein from an antibody column. Both efficiency of purification and efficiency of proteolytic cleavage of receptor from MBP may be tested for each construct.

A more interesting approach involves the placement of affinity tag within the body of maltose binding protein. MBP is highly tolerant to multiple amino acid deletions and insertions in several positions (Duplay et al. 1987). These sites have been mapped and extensively characterized (Betton et al. 1993). Two positions, one at residue 133 and the other at 303, are particularly interesting because they are entirely solvent accessible and are located at a considerable distance from the C-terminus of MBP, where the receptor is fused. Antigen epitopes inserted at these loci have shown efficient recognition by the corresponding antibodies. MBP mutants with several different affinity tags inserted at these positions in MBP may be generated. Given the distance of these regions from the site of the fusion between MBP and the receptor, antibody binding to the internal antigen tags may be compatible with specific proteolysis of the engineered protease sites between MBP and the receptor, allowing elution of the unfused receptor from the antibody resin.

The reconstitution of detergent-solubilized protein into the lipid bilayers, typically in liposomes, can be used to verify structural integrity and to screen for any lipids optimal for activity and perhaps for crystallization (Cerione et al. 1983). Lipids have proved critical to the formation of many highly ordered crystals of membrane proteins, and reconstitution provides an excellent way to determine which lipid or class of lipids promotes maximal activity of the receptor. Receptor purified in the detergent system described herein for the bacterial system (dodecyl maltoside+cholesteryl hemisuccinate) can be used to reconstitute in vesicles made from a variety of natural lipid sources (e.g. E. coli, bovine brain, egg) and then tested by radioactive ligand binding assay for activity. Using the reconstitution assay the detergent screen can be repeated to discover any additional detergents missed during the initial screen which could extract receptor in a functional state. Some detergents cause structural perturbations to the receptor which can drastically alter ligand affinity. Nevertheless, they may sufficiently preserve structure enough to allow physiological activity to be recovered following lipid reconstitution and detergent removal. Such detergents may also be amenable to crystallization and provide meaningful structural data about the protein.

Reconstitution could also afford an excellent way to concentrate and purify the receptor. Following reconstitution, protein could then be extracted in a pure state from the bilayer at various detergent:lipid concentrations to find the right balance between the solubility and structural integrity suitable for crystallization. The eukaryotic membrane protein structures which have been solved to date have come from the direct detergent solubilization of protein from naturally abundant and pure sources (Shinzawa-Itoh et al. 1995; Toyoshima et al. 2000; Palczewski et al. 2000). An authentic reconstitution membrane could simulate this natural environment.

Receptor Complexes Stabilized with Protein Ligands

Proteins that interact with other molecules usually are stabilized by the interaction. Crystal structures of complexes often show lower flexibility than those for corresponding apo states, for example. In reverse, it can be expected that membrane proteins may lose stability when removed from their natural lipid bilayer environment into a detergent miscelle. There is a large entropic penalty for the formation of crystal contacts involving flexible regions. Thus, such regions tend to be excluded from contact and crystallization probability is sharply reduced when a substantial portion of the molecular surface is flexible (Kwong et al. 1999). In the case of solubilized membrane proteins, where substantial fractions of the surface are inherently flexible, this becomes an important factor. A strategy for using antibody fragments in membrane protein crystallization has been expounded (Hunte et al. 2002) and has been applied with good effect (Iwata et al. 1995; Ostermeier et al. 1997; Zhou et al. 2001). Another example comes from attempts to crystallize HIV gp120 (Kwong et al. 1999). Crystals were obtained when flexible loops were removed to make core constructs, deglycosylated to remove further conformational heterogeneity, and stabilized the protein with CD4 and antibody ligands.

A polyclonal antibody raised against a synthetic peptide corresponding to the extracellular 4-5 loop of the olfactory receptor SP1 has been shown to recognize this receptor by immuno-staining and immuno-precipitation under non-denaturing conditions from solubilized nasal tissue. A peptide corresponding to the same region of 5HT2c has been synthesized. As a first step towards the production of monoclonal antibodies, mice have been immunized with the SP1 and 5HT2c peptides. Serum collected from mice immunized with SP1 peptide has been shown to immuno-precipitate SP1 expressed in E. coli (details of the bacterial expression are discussed below). Two glycosylation sites are in the extracellular 4-5 loop of both rat and mouse 5HT2c. Therefore, it is possible that the corresponding (unglycosylated) peptide may elicit an immune response. Following a similar reasoning, if one or more monoclonal antibodies are obtained in this way they may be useful only for 5HT2c expressed in bacteria.

Expression Stabilized by Fusions with Gα and Other Partners

In vivo the 5HT2c receptor interacts with Gαq. The last 11-amino acid residues of the alpha subunit of the G-protein heterotrimer are known to be critical for the interaction with its cognate receptors (Martin et al. 1996). A chimeric Gα construct, called GαiqC, was made in which the C-terminal 11 residues of Gαi were replaced with those of Gαq. This protein was engineered because functional Gαi can be expressed in bacteria and Gαi has proven to be more structurally tractable than Gαq. The 5HT2c receptor was genetically fused to these two Gα subunits. Fusions were made to the full length receptor and also, as in the robustly expressing fusions of 5HT2c to TRX, to 5HT2c truncated at residue 402. Twenty different constructs were generated, in which 5 linkers were tried for each of the 4 combinations of receptor and Gα. These MBP-5HT2c-Gα fusions were expressed in E. coli following the same protocol used for the MBP-5HT2c constructs. Regarding expression and relative specific activity of MBP-5HT2c-Gαq and MBP-5HT2c-GαiqC fusions in comparison to those of MBP-5HT2c and MBP-5HT2c(402)-TRX, and as observed before, expression levels seem to correlate rather well with activity data. Although these fusions do not express as well as MBP-5HT2c(402)-TRX some express substantially better than MBP-5HT2c. The length of the linker seems to be an important factor. Moreover, comparison of the intensity of the 5HT2c degradation band between different constructs shows that in some cases the presence of the Gα subunit is protecting if not indeed stabilizing the receptor.

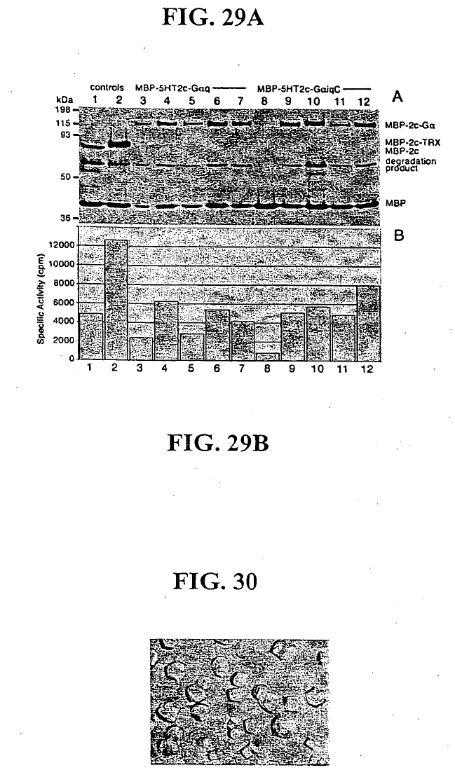

FIGS. 29A and 29B show expression and activity of C-terminal fusions to 5HT2c. FIG. 29A shows quantitative western blot analysis of MBP-5HT2c-Gαq (lanes 3 to 7) and MBP-5HT2c-GαiqC fusions (lanes 8 to 12) compared to MBP-5HT2c (lane1) and MBP-5HT2c-TRX (lane 2). For each set of fusion with a given Gα, the linker with the receptor was increased in length progressively (from lane 3 to 7, and from lanes 8 to 12). FIG. 29B shows relative specific activity data, measured at 2 nM 3H-LSD with and without 10 mM mesulergine. Equal numbers of spheroplasts were assayed, and assays were performed in triplicate.

There is a rich literature on GPCR-GA fusions (Seifert et al. 1999). A series of Gα fusions to the 5HT2c receptor have been generated and tested, and a library of fusions between a series of serotonin receptors and their cognate G proteins is in production. These receptor-Gα pairs are 5HT2c-Gαq (which were also emulated with GiαqC), 5HT1a-Gαi, and 5HT7-Gas. Five linkers of different lengths and composition are being tested for each of the pairs in assays of expression and ligand-binding activity. Experiments are in the context of the MBP fusions for expression. Comparisons can be made to the well characterized expression pattern for the MBP-5HT and the MBP-5HT2c(402)-TRX fusions. Besides the aim of achieving stabilization, these or follow-up experiments with other serotonin sub-family members (FIG. 7) may ultimately yield structural information about signaling through all three major classes of G proteins.

In view of evidence that one or more of the fusion constructs are functional they may be screened for suitable extraction conditions. Different detergents as well as stabilizing additives, such as lipids and cholesterol derivatives, can be screened, and extraction efficiency and ligand-binding activity can be assayed. Functionality of the detergent-solubilized species can be further assessed by probing its ability to interact with the G dimer. The β1γ2 isoform of the dimer can be produced in baculovirus-infected insect cells following well established protocols (Ueda et al. 1994). This isoform shows broad promiscuity in its interaction with different G subunits. The G-protein heterotrimer can be stably docked to the activated cognate GPCR when nucleotide free, and eluted by addition of GTP (Brown et al. 1993; Knezevic et al. 1993; Santos-Alvarez et al. 2000).

The formation of the quaternary complex (receptor-G fusion and Gβdimer) can be assayed initially by size-exclusion chromatography and native-gel electrophoresis. One could also test complex assembly by a co-immunoprecipitation experiment wherein one of the components (either the receptor-Gα fusion or Gβγ dimer) is bound to an inert support matrix (Pang et al. 1989). If complex formation is successful, the components co-elute from the column. In the case of GPCRs, specific dissociation of the purified complex can be achieved by addition of receptor agonist and GTP. Functionality of both partners in the fusion is paramount. By assaying the incorporation of radiolabelled GTPγs (a non-hydolyzable analogue of GTP), upon addition of agonist (serotonin) in the presence of Gβγ it can be determined if both receptor and G-protein are functional (Pang et al. 1989; Wurch et al. 2001). An ultimate test of functionality is to generate a proteolytically cleavable receptor-Gα fusion and to determine whether the quaternary complex reassembles with Gαβγ after protease treatment. Size-exclusion chromatography may be the most efficient way to address this question.

Various efforts may be made to raise monoclonal antibodies against conformational epitopes. A parallel effort has been made with a synthetic constrained peptide corresponding to an external loop from the 5HT2c receptor. This peptide may elicit an immunogenic-response, and the resulting monoclonal antibody-producing cells can be screened by non-denaturing ELISA (Padan et al. 1998) and immunoprecipitation. Alternative approaches to antibody generation include immunizing mice with detergent-solubilized purified protein, with protein that has been reconstituted in a lipid bilayer, with bacterial spheroplasts, or cells.

Crystallization of 5HT2c Produced in Mammalian Cells

Crystallization experiments were performed with the material purified as described above. Commercially-available screens as well as in-house screens were tested (see description below). Different temperatures were screened, and crystallization trials set up at 4° C. were found to yield the most promising results. Different ligands were also screened for their ability to promote crystallization. Mesulergine and serotonin were amongst the ligands screened. Polyethylene glycol precipitants on average gave better results than salt-based crystallization conditions. So far only small (<10 μM) crystals have been obtained. Although too small to perform meaningful diffraction experiments, pools of crystals were isolated, washed, run on a denaturing gel and western blotted. These experiments suggested that, at least for some conditions, the crystals did contain 5HT2c receptor.

Crystallization of 5HT2c Fusion Protein

Uncleavable fusions of MBP and 5HT2c prepared as described above were used for crystallization experiments. The majority of these experiments were performed using in-house designed screens. The design of the crystallization screens began with the assumption that the behavior of protein-detergent complexes is driven primarily by the properties of the detergent micelles alone. Detergents exhibit a property known as the “cloud point”, at which they undergo a phase transition that partitions the detergent into two separate detergent-rich and detergent-poor phases. If detergent-protein complexes are approximately like detergent micelles, then the location of this detergent-only phase transition in n-dimensional crystallization space should be located near the boundary of crystallization for the detergent-solubilized membrane protein.

A set of potentially useful detergents (several maltosides and several glycosides) initially were screened, at a given useful concentration above the critical micellar concentration (CMC), against a series of precipitants at increasing concentrations. From this simple mono-dimensional screen, the concentration at which a given precipitant-detergent combination exhibits phase separation can be estimated. To the positives from the initial screen (which are referred herein as “PF1”) several other variables such as pH buffers and a series of common secondary precipitants at different concentrations were added to generate PF2, the second generation screen. It was assumed that the addition of buffers and other precipitants at relatively low concentrations would not drastically shift the phase transition boundary of the detergent. Purified, detergent-solubilized protein was then used at this stage to determine the initial effectiveness of this approach. Against this second generation screen, an encouraging number of conditions yielded protein precipitates.

The hits from the second screen were then expanded and the concentrations of components were systematically screened more finely to optimize the crystallization parameters. The character of the protein precipitate improved with each generation screen. Iteration of this process eventually led to a sixth generation crystallization screen (PF6) which yielded protein crystals of promising morphology (FIG. V) which were subject to x-ray diffraction analysis (FIG. W). The rationale behind this approach, that solubilized membrane proteins should crystallize near the phase transition boundary of their detergent components, has also been used with considerable success by others (Loll et al. 2001; Song et al. 1998; Garavito et al. 1996).

FIG. 30 shows crystals of the MBP-serotonin receptor fusion protein. Typical crystals of this kind have dimensions of 80 μM×80 μM×30 μM. These crystals were generated using the PF6 screen.

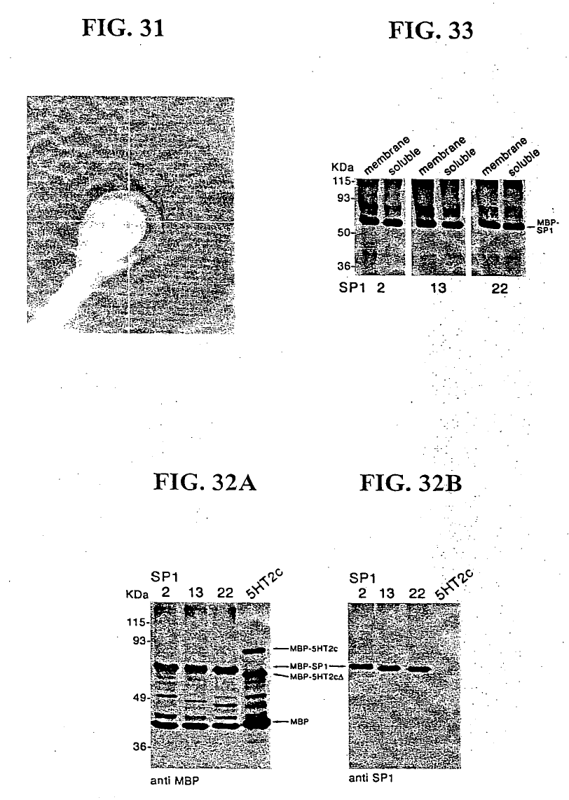

FIG. 31 shows a diffraction pattern of a crystal obtained using the PF6 screen. The diffraction experiment was performed on NSLS beamline X4A at Brookhaven National Laboratory. Crystals were frozen in liquid nitrogen prior to the experiment. Diffraction could be observed to spacings corresponding to 9 Å resolution.

The discussion above of crystallizing bacterially expressed 5HT2c receptor centers on constructs in which MBP and the receptor are fused directly without a proteolytically cleavable linker. Bacterially expressed material may include proteins in which the MBP has been cleaved prior to crystallization. Constructs in which MBP and 5HT2c are separated by a proteolytically-cleavable linker have already been generated and tested for expression, activity and efficiency of cleavage.

Receptor complexes with stabilizing ligands have been produced, and have an enhanced probability for crystallization. The majority of membrane protein structures solved to date have demonstrated a specific lipid requirement and in many of the structures, the electron density of a specific lipid molecule has been observed (Valiyaveetil et al. 2002). The lipid requirements of the 5HT2c receptor for crystallization can be investigated by extracting the purified receptor from reconstituted lipid bilayers made using a variety of individual lipids and lipid mixtures. Extraction can be done with various detergents at different concentrations to determine the optimal detergent:lipid ratio. Crystallization may also be attempted in cubo. Some lipids naturally form three-dimensionally ordered structures which can be used as a platform to induce crystallographic contacts of membrane proteins incorporated into these lipid structures. These cubic lipidic phases have been used to successfully crystallize a variety of retinal-conjugated bacterial membrane proteins (Landau 1996).

Crystals that are grown can be tested for diffraction quality by exposure to x-ray beams, such as at the synchrotron facilities at Brookhaven and Argonne National Laboratories. The testing may focus appropriately cryo-preserved samples, but capillary mounts may also be used to test intrinsic diffraction quality. It may be desired to obtain crystals that diffract sufficiently well to permit the construction of atomic-level models, which means diffraction at least as far as 3.5 Å and preferably better. Given similarities with rhodopsin, it may be possible to use the method of molecular replacement for structure determination, but experimental phases from the methods of multiple isomorphous replacement (MIR) or multiwavelength anomalous diffraction (MAD) may prove advantageous. In this regard, the bacterial expression system provides the now routine possibility for incorporation of selenomethionine for MAD analysis (Hendrickson et al. 1990; Hendrickson 1991).

The methodologies described herein can be extended to other members of the serotonin GPCR family as well as other GPCRs.

Olfactory Receptors

Olfactory receptors represent the most abundant class of GPCRs. These proteins are particularly resilient to expression in eukaryotic cells. The absence of identifiable high-affinity ligands for the majority of these proteins also poses a disadvantage in attempting to characterize these molecules biochemically and structurally. Nevertheless, the expression of an olfactory receptor was attempted as a fusion to MBP in bacteria. A particular receptor of murine origin named SP1 was chosen, a choice based mainly on the availability of a specific antibody. In an approach similar to the one followed for the expression of serotonin receptors, MBP was genetically fused to three different positions along the N-terminus of this receptor. Similar expression protocols to those used to express serotonin receptors were followed. FIGS. 32A and 32B correspond to expression of olfactory receptor SP1. FIG. 32A shows a western blot probed with anti-MBP antibody. The first three lanes, marked 2, 13 and 22, show expression of MBP fused to SP1 at positions 2, 13 and 22 respectively. The last lane, marked 5HT2c, shows expression of the MBP-5HT2c receptor fusion. FIG. 32B shows a western blot probed with anti-SP1 antibody. Markings are as in FIG. 32A. Equal numbers of cells were loaded in each lane. MBP-5HT2cA shows a degradation product of the MBP-5HT2c fusion. FIGS. 32A and 32B show that all three constructs could be expressed at levels comparable to those of 5HT2c receptor. Integrity of the expressed fusion protein was verified by western blot probing with both an anti-MBP antibody and the anti-SP1 antibody.

In order to further assess the quality of the expressed fusion protein, bacterial membranes were generated. The receptor was found to be associated entirely with the membrane fraction.

FIG. 33 corresponds to detergent solubilization of olfactory receptor SP1. Western blots are probed with anti-SP1 antibody. The three panels marked 2, 13 and 22 refer to the proteins generated by the fusion of MBP to SP1 at positions 2, 13 and 22 respectively. In each panel, the first lane represents membranes, the second lane material extracted from these membranes with dodecyl-β-maltoside. As shown in FIG. 33, the receptor could be quantitatively extracted from these membranes with a non-ionic detergent. These data are by no means a proof of correct folding of this protein, but nevertheless they do demonstrate that the receptor is associated with the membrane as opposed to being expressed in the form of inclusion bodies.

Glycoprotein Hormone Receptors

Existing studies have generated a longstanding interest in glycoprotein hormone receptors, having determined the structure of human chorionic gonadotropin (hCG) (Kwong et al. 1998) and made an analysis of hormone binding to the large, extracellular leucine-rich repeat domains of these receptors (Jiang et al. 1995). The model produced from the studies suggests that a loop from the hormone α-chain may interact with the transmembrane GPCR domain to activate the receptor. Complexes of hCG with the extracellular portion of human leuteinizing hormone receptor (LHR) and also of follicle stimulating hormone (FSH) complexed with the extracellular domain of its FSH receptor have been produced. Crystals have been grown in each case, and efforts are ongoing to improve protein supplies and diffraction characteristics of the crystals.

Chemokine Receptors Co-Opted by HIV

Studies on the interaction of the external envelope glycoprotein with its cellular receptors have yielded structures of gp120 complexed with the D1D2 domain of CD4 and the Fab portion of a neutralizing antibody that interacts with chemokine co-receptors. The structures include complexes of both a laboratory-adapted strain (Kwong et al. 1998), which interacts preferentially with the CXCR4 chemokine receptor, and also a primary isolate (Kwong et al. 1998), which interacts with CCR5. Procedures for the enhanced expression of CCR5 in mammalian cells (Mirzabekov et al. 1999) have been developed.

### REFERENCES FOR FIRST SET OF EXPERIMENTAL DETAILS

Henderson, R., and Unwin, P. N. (1975) “Three-dimensional model of purple membrane obtained by electron microscopy”, Nature, 257:28-32.

Ross, E. M., and Gilman, A. G. (1977) “Resolution of some components of adenylate cyclase necessary for catalytic activity”, J Biol Chem, 252:6966-6969.

Ovchinnikow, Y. A., Abdulaev, N. G., Dergachev, A. E., Drachev, A. L., Drachev, L. A., Kaulen, A. D., Khitrina, L. V., Lazarova, Z. P. and Skulachev, V. P. (1982) “Photoelectric and spectral responses of bacteriorhodospin modified by carbdiimide and amine derivatives”, Eur. J. Biochem, 127:325-332.

Thomas, D. D. and Stryer, L. (1982) “Transverse location of the retinal chromophore of rhodopsin in rod outer segment disc membranes”, J Mol Biol, 154:145-157.

Cerione, R. A., Strulovici, B., Benovic, J. L., Strader, C. D., Caron, M. G., and Lefkowitz, R. J. (1983) “Reconstitution of beta-adrenergic receptors in lipid vesicles: affinity chromatography-purified receptors confer catecholamine responsiveness on a heterologous adenylate cyclase system”, Proc Natl Acad Sci USA, 80:4899-4903.

Hargrave, P. A., McDowell, J. H., Curtis, D. R., Wang, J. K., Juszczak, E., Fong, S. L., Rao, J. K., and Argos, P. (1983) “The structure of bovine rhodopsin” Biophys Struct Mech, 9:235-244.

Nathans, J., and Hogness, D. S. (1983) “Isolation, sequence analysis, and intron-exon arrangement of the gene encoding bovine rhodopsin”, Cell, 34:807-814.

Deisenhofer, J., Epp, O., Miki, K., Huber, R., and Michel, H. (1985) “Structure of the protein subunits in the photosynthetic reaction center of Rhodopseudomonas viridis at 3A resolution”, Nature, 318:618-624.

Dixon, R. A., Kobilka, B. K., Strader, D. J., Benovic, J. L., Dohlman, H. G., Frielle, T., Bolanowski, M. A., Bennett, C. D., Rands, E., Diehl, R. E., et al. (1986) “Cloning of the gene and cDNA for mammalian beta-adrenergic receptor and homology with rhodopsin”, Nature, 321:75-79.

Stryer, L. (1986) “Cyclic GMP cascade of vision”, Annu Rev Neurosci, 9:87-119.

Duplay, P., Szmelcman, S., Bedouelle, H., and Hofnung, M. (1987) “Silent and functional changes in the periplasmic maltose-binding protein of Escherichia coli K12. I. Transport of maltose”, J Mol Biol, 194:663-673.

Gilman, A. G. (1987) “G proteins: transducers of receptor-generated signals”, Annu Rev Biochem, 56:615-649.

Kobilka, B. K., Frielle, T., Collins, S., Yang-Feng, T., Kobilka, T. S., Francke, U., Lefkowitz, R. J., and Caron, M. G. (1987) “An intronless gene encoding a potential member of the family of receptors coupled to guanine nucleotide regulatory proteins”, Nature, 329:75-79.

D. Julius, A. B. MacDermott, R. Axel and T. M. Jessell (1988) “Molecular characterization of a functional cDNA encoding the serotonin 1c receptor”, Science, 241:558-564.

D. Julius, T. J. Livelli, T. M. Jessell and R. Axel (1989) “Ectopic expression of the serotonin 1c receptor and the triggering of malignant transformation”, Science, 244:1057-1062.

Pang, I. H., and Sternweis, P. C. (1989) “Isolation of the alpha subunits of GTP-binding regulatory proteins by affinity chromatography with immobilized beta gamma subunits“, Proc Natl Acad Sci USA, 86:7814-7818.

J. Sambrook et al. (1989) Molecular Cloning: a laboratory manual. Second Edition, Cold Spring Harbor Laboratory Press.

Henderson, R., Baldwin, J. M., Ceska, T. A., Zemlin, F., Beckmann, E., and Downing, K. H. (1990) “Model for the structure of bacteriorhodopsin based on high-resolution electron cryo-microscopy”, J Mol Biol, 213:899-929.

Hendrickson W., Horton J., and LeMaster, D. (1990) “Selenomethionyl proteins produced for analysis by multiwavelength anomalous diffraction (MAD): a vehicle for direct determination of three-dimensional structure”, Embo J, 9:1665-1672.

Ryu, S., Kwong, P., Truneh, A., Porter, T., Arthos, J., Rosenberg, M., Dai, X., Xuong, N.-h., Axel, R., Sweet, R., and Hendrickson, W. (1990) “Crystal Structure of an HIV-binding Recombinant Fragment of Human CD4”, Nature, 348:419-426.

Buck, L. and Axel, R. (1991) “A novel multigene family may encode odorant receptors: a molecular basis for odor recognition”, Cell, 65:175-187.

Hendrickson, W. (1991) “Determination of macromolecular structures from anomalous diffraction of synchrotron radiation”, Science, 254:51-58.

Reilander, H., Boege, F., Vasudevan, S., Maul, G., Hekman, M., Dees, C., Hampe, W., Helmreich, E. J., and Michel, H. (1991) “Purification and functional characterization of the human beta 2-adrenergic receptor produced in baculovirus-infected insect cells”, FEBS Lett, 282:441-444.

Spurlino, J. C., Lu, G. Y., and Quiocho, F. A. (1991) “The 2.3-A resolution structure of the maltose- or maltodextrin-binding protein, a primary receptor of bacterial active transport and chemotaxis”, J Biol Chem, 266:5202-5219.

D. J. Leahy, R. Axel and W. A. Hendrickson (1992) “Crystal structure of a soluble form of the human T cell coreceptor CD8 at 2.6 Å resolution”, Cell, 68:1145-1162.

Probst, W. C., Snyder, L. A., Schuster, D. I., Brosius, J., and Sealfon, S. C. (1992) “Sequence alignment of the G-protein coupled receptor superfamily”, DNA Cell Biol, 11:1-20.

Betton, J. M., Martineau, P., Saurin, W., and Hofnung, M. (1993) “Location of tolerated insertions/deletions in the structure of the maltose binding protein”, FEBS Lett, 325:34-38.

Brown, P. J., and Schonbrunn, A. (1993) “Affinity purification of a somatostatin receptor-G-protein complex demonstrates specificity in receptor-G-protein coupling”, J Biol Chem, 268:6668-6676.

Conklin, B. R. and Bourne, H. R. (1993) “Structural elements of G alpha subunits that interact with G beta gamma, receptors, and effectors”, Cell, 73:631-641.

Grisshammer, R., Duckworth, R., and Henderson, R. (1993) “Expression of a rat neurotensin receptor in Escherichia coli”, Biochem J, 295:571-576.

Knezevic, I., Borg, C., and Le Breton, G. C. (1993) “Identification of Gq as one of the G-proteins which copurify with human platelet thromboxane A2/prostaglandin H2 receptors”, J Biol Chem, 268:26011-26017.

Noel, J. P., Hamm, H. E., and Sigler, P. B. (1993) “The 2.2 A crystal structure of transducin-alpha complexed with GTP gamma S”, Nature, 366:54-663.

Zhang, D., and Weinstein, H. (1993) “Signal transduction by a 5-HT2 receptor: a mechanistic hypothesis from molecular dynamics simulations of the three-dimensional model of the receptor complexed to ligands”, J Med Chem, 36:934-938.

Coleman, D. E., Berghuis, A. M., Lee, E., Linder, M. E., Gilman, A. G., and Sprang, S. R. (1994) “Structures of active conformations of Gi alpha 1 and the mechanism of GTP hydrolysis”, Science, 265:1405-1412.

Hubbard, S. R., Wei, L., Ellis, L., and Hendrickson, W. A. (1994) “Crystal structure of the tyrosine kinase domain of the human insulin receptor”, Nature, 372:746-754.

Picot, D., Loll, P. J., and Garavito, R. M. (1994) “The X-ray crystal structure of the membrane protein prostaglandin H2 synthase-1”, Nature, 367:243-249.

Ryu S., Truneh A., Sweet, R., and Hendrickson W. (1994) “Structures of an HIV and MHC binding fragment from human CD4 as refined in two crystal lattices”, Structure, 2:59-74.

Strader, C. D., Fong, T. M., Tota, M. R., Underwood, D., and Dixon, R. A. (1994) “Structure and function of G protein-coupled receptors”, Annu Rev Biochem, 63:101-132.

Ueda, N., Iniguez-Lluhi, J. A., Lee, E., Smrcka, A. V., Robishaw, J. D., and Gilman, A. G. (1994) “G protein beta gamma subunits. Simplified purification and properties of novel isoforms”, J Biol Chem, 269:4388-4395.

Wu H., Lustbader J., Liu Y., Canfield R., and Hendrickson W. (1994) “Structure of human chorionic gonadotropin at 2.6 A resolution from MAD analysis of the selenomethionyl protein”, Structure, 2:545-558.

Abramowski, D., and Staufenbiel, M. (1995) “Identification of the 5-hydroxytryptamine2C receptor as a 60-kDa N-glycosylated protein in choroid plexus and hippocampus”, J Neurochem, 65:782-790.

Backstrom, J. R., Westphal, R. S., Canton, H., and Sanders-Bush, E. (1995) “Identification of rat serotonin 5-HT2C receptors as glycoproteins containing N-linked oligosaccharides”, Brain Res Mol Brain Res, 33:311-318.

Grisshammer, R., and Tate, C. G. (1995) “Overexpression of integral membrane proteins for structural studies”, Q Rev Biophys, 28:315-422.

Iwata, S., Ostermeier, C., Ludwig, B., and Michel, H. (1995) “Structure at 2.8 A resolution of cytochrome c oxidase from Paracoccus denitrificans”, Nature, 376:660-669.

Jiang X., Dreano M., Buckler D., Cheng S., Ythier A., Wu H., Hendrickson W., and el Tayar, N. (1995) “Structural predictions for the ligand-binding region of glycoprotein hormone receptors and the nature of hormone-receptor interactions”, Structure, 3:1341-1353.

McDonald, N. Q., Panayotatos, N., and Hendrickson, W. A. (1995) “Crystal structure of dimeric human ciliary neurotrophic factor determined by MAD phasing”, Embo J, 14:2689-2699.

Shinzawa-Itoh, K., Ueda, H., Yoshikawa, S., Aoyama, H., Yamashita, E., and Tsukihara, T. (1995) “Effects of ethyleneglycol chain length of dodecyl polyethyleneglycol monoether on the crystallization of bovine heart cytochrome c oxidase”, J Mol Biol, 246:572-575.

Wall, M. A., Coleman, D. E., Lee, E., Iniguez-Lluhi, J. A., Posner, B. A., Gilman, A. G., and Sprang, S. R. (1995) “The structure of the G protein heterotrimer Gi alpha 1 beta 1 gamma 2”, Cell, 83:1047-1058.

Weiss, H. M., Haase, W., Michel, H., and Reilander, H. (1995) “Expression of functional mouse 5-HT5A serotonin receptor in the methylotrophic yeast Pichia pastoris: pharmacological characterization and localization”, FEBS Lett, 377:451-456.

Garavito, R. M., Picot, D., and Loll, P. J. (1996) “Strategies for crystallizing membrane proteins”, J Bioenerg Biomembr, 28:13-27.

Kurumbail, R. G., Stevens, A. M., Gierse, J. K., McDonald, J. J., Stegeman, R. A., Pak, J. Y., Gildehaus, D., Miyashiro, J. M., Penning, T. D., Seibert, K., Isakson, P. C., and Stallings, W. C. (1996) “Structural basis for selective inhibition of cyclooxygenase-2 by anti-inflammatory agents”, Nature, 384:644-648.

Lambright, D. G., Sondek, J., Bohm, A., Skiba, N. P., Hamm, H. E., and Sigler, P. B. (1996) “The 2.0 A crystal structure of a heterotrimeric G protein”, Nature, 379:311-319.

Landau, E. M., and Rosenbusch, J. P. (1996) “Lipidic cubic phases: a novel concept for the crystallization of membrane proteins”, Proc Natl Acad Sci USA, 93:14532-14535.

Martin, E. L., Rens-Domiano, S., Schatz, P. J., and Hamm, H. E. (1996) “Potent peptide analogues of a G protein receptor-binding region obtained with a combinatorial library”, J Biol Chem, 271:361-366.

Sondek, J., Bohm, A., Lambright, D. G., Hamm, H. E., and Sigler, P. B. (1996) “Crystal structure of a G-protein beta gamma dimer at 2.1 A resolution”, Nature, 379:369-374.

Tsukihara, T., Aoyama, H., Yamashita, E., Tomizaki, T., Yamaguchi, H., Shinzawa-Itoh, K., Nakashima, R., Yaono, R., and Yoshikawa, S. (1996) “The whole structure of the 13-subunit oxidized cytochrome c oxidase at 2.8 A”, Science, 272:1136-1144.

Tucker, J., and Grisshammer, R. (1996) “Purification of a rat neurotensin receptor expressed in Escherichia coli”, Biochem J, 317:891-899.

Wu H., Myszka D., Tendian S., Brouillette, C., Sweet, R., Chaiken, I., and Hendrickson, W. (1996) “Kinetic and structural analysis of mutant CD4 receptors that are defective in HIV gpl20 binding”, Proc Natl Acad Sci USA, 93:15030-15035.

Yamaguchi H, and Hendrickson W (1996) “Structural basis for activation of human lymphocyte kinase Lck upon tyrosine phosphorylation”, Nature, 384:484-489.

F. Yang, L. G. Moss and G. N. Phillips (1996) “The Molecular Structure of Green Fluorescent Protein”, Nature Biotech., 14:1219-1220.

J. M. Baldwin, G. F. X. Schertler and V. M. Unger (1997) “An alpha-carbon template for the transmembrane helices in the rhodopsin family of G-protein-coupled receptors”, J. Mol. Biol., 272:144-164.

Ostermeier, C., and Michel, H. (1997) “Crystallization of membrane proteins”, Curr Opin Struct Biol, 7:697-701.

Ostermeier, C., Harrenga, A., Ermler, U., and Michel, H. (1997) “Structure at 2.7 A resolution of the Paracoccus denitrificans two-subunit cytochrome c oxidase complexed with an antibody FV fragment”, Proc Natl Acad Sci USA, 94:10547-10553.

L. Song and J. E. Gouaux (1997) “Membrane protein crystallization: application of sparse matrics to the a-hemolysin heptamer”, Meth. Enzymol., 276:60-74.

Tesmer, J. J., Sunahara, R. K., Gilman, A. G., and Sprang, S. R. (1997) “Crystal structure of the catalytic domains of adenylyl cyclase in a complex with Gsalpha.GTPgammaS”, Science, 278:1907-1916.

V. M. Unger, P. A. Hargrave, J. M. Baldwin and G. F. X. Schertler (1997) “Arrangement of rhodopsin transmembrane a-helices”, Nature, 389:203-206.

Wu, H., Kwong, P. D., and Hendrickson, W. A. (1997) “Dimeric association and segmental variability in the structure of human CD4”, Nature, 387:527-530.

DiGabriele A., Lax I., Chen D., Svahn C., Jaye M., Schlessinger, J., and Hendrickson, W. (1998) “Structure of a heparin-linked biologically active dimer of fibroblast growth factor”, Nature, 393:812-817.

Doyle, D. A., Morais, C. J., Pfuetzner, R. A., Kuo, A., Gulbis, J. M., Cohen, S. L., Chait, B. T., and MacKinnon, R. (1998) “The structure of the potassium channel: molecular basis of K+ conduction and selectivity”, Science, 280:69-77.

Kwong, P., Wyatt, R., Robinson, J., Sweet, R., Sodroski, J., and Hendrickson, W. (1998) “Structure of an HIV gpl20 envelope glycoprotein in complex with the CD4 receptor and a neutralizing human antibody”, Nature, 393:648-659.

Padan, E., Venturi, M., Michel, H., and Hunte, C. (1998) “Production and characterization of monoclonal antibodies directed against native epitopes of NhaA, the Na+/H+ antiporter of Escherichia coli”, FEBS Lett, 441:53-58.

Song, L., and Gouauex, E. (1998) “Crystallization of the alpha-hemolysin heptamer solubilized in decyldimethyl- and decyldiethylphosphine oxide”, Acta Crystallogr D Biol Crystallogr, 54:276-278.

Wallin, E., and von Heijne, G. (1998) “Genome-wide analysis of integral membrane proteins from eubacterial, archaean, and eukaryotic organisms”, Protein Sci, 7:1029-1038.

Weiss, H. M., Haase, W., Michel, H., and Reilander, H. (1998) “Comparative biochemical and pharmacological characterization of the mouse 5HT5A 5-hydroxytryptamine receptor and the human beta2-adrenergic receptor produced in the methylotrophic yeast Pichia pastoris”, Biochem J, 330:1137-1147.

Barnes, N. M., and Sharp, T. (1999) “A review of central 5-HT receptors and their function”, Neuropharmacology, 38:1083-1152.

Grisshammer, R., Averbeck, P., and Sohal, A. K. (1999) “Improved purification of a rat neurotensin receptor expressed in Escherichia coli”, Biochem Soc Trans, 27:899-903.

Kwong, P. D., Wyatt, R., Desjardins, E., Robinson, J., Culp, J., Hellmig, B., Sweet, R., Sodroski, J., and Hendrickson, W. (1999) “Probability analysis of variational crystallization and its application to gpl20, the exterior envelope glycoprotein of type 1 human immunodeficiency virus (HIV-1)”, J Biol Chem, 274:4115-4123.

Mirzabekov, T., Bannert, N., Farzan, M., Hofmann, W., Kolchinsky, P., Wu, L., Wyatt, R., and Sodroski, J. (1999) “Enhanced expression, native purification, and characterization of CCR5, a principal HIV-1 coreceptor”, J Biol Chem, 274:28745-28750.

Seifert, R., Wenzel-Seifert, K., and Kobilka, B. K. (1999) “GPCR-Galpha fusion proteins: molecular analysis of receptor-G-protein coupling”, Trends Pharmacol Sci, 20:383-389.

Armstrong, N., and Gouaux, E. (2000) “Mechanisms for activation and antagonism of an AMPA-sensitive glutamate receptor: crystal structures of the GluR2 ligand binding core”, Neuron, 28:165-181.

Furukawa, H., and Haga, T. (2000) “Expression of functional M2 muscarinic acetylcholine receptor in Escherichia coli”, J Biochem (Tokyo), 127:151-161.

W. Hampe, R.-H. Voss, W. Haase, F. Boege, H. Michel and H. Reilander (2000) “Engineering of a proteolytically stable human b2-adrenergic receptor/maltose-binding protein fusion and production of the chimeric protein in Escherichia coli and baculovirus-infected insect cells”, J. Biotechnology, 77:219-234.

Hendrickson, W. A., and Wuthrich, K. (2000) Macromolecular Structures 2000, Current Opinion.

Jiang, X., Gurel, O., Mendiaz, E., Stearns, G., Clogston, C., Lu, H., Osslund, T., Syed, R., Langley, K., and Hendrickson, W. (2000) “Structure of the active core of human stem cell factor and analysis of binding to its receptor kit”, Embo J, 19:3192-3203.

Kandel, E. R., Schwartz, J. H., and Jessel, T. M. (2000) Principles of neural science, McGraw-Hill, New York. Kwong, P., Wyatt, R., Majeed, S., Robinson J., Sweet, R., Sodroski, J., and Hendrickson, W. (2000) “Structures of HIV-1 gpl20 envelope glycoproteins from laboratory-adapted and primary isolates”, Structure Fold Des, 8:1329-1339.

Okada, T., Le Trong, I., Fox, B. A., Behnke, C. A., Stenkamp, R. E., and Palczewski, K. (2000) “X-Ray diffraction analysis of three-dimensional crystals of bovine rhodopsin obtained from mixed micelles”, J Struct Biol, 130:73-80.

Loll, P. J., Allaman, M., and Wiencek, J. (2001) “Assessing the role of detergent-detergent interactions in membrane protein crystallization”, Journal of Crystal Growth, 232:432-438.

K. Palczewski, T. Kumasaka, T. Hori, C. A. Behnke, H. Motoshima, B. A. Fox, I. Le Trong, D. C. Teller, T. Okada, R. E. Stenkamp, M. Yamamoto and M. Miyano (2000) “Crystal structure of rhodopsin: a G protein-coupled receptor”, Science, 289:739-745.

Santos-Alvarez, J., and Sanchez-Margalet, V. (2000) “Affinity purification of pancreastatin receptor-Gq/11 protein complex from rat liver membranes”, Arch Biochem Biophys, 378:151-156.

Stauber, D., DiGabriele, A., and Hendrickson, W., (2000) “Structural interactions of fibroblast growth factor receptor with its ligands”, Proc Natl Acad Sci USA, 97:49-54.

Toyoshima, C., Nakasako, M., Nomura, H., and Ogawa, H. (2000) “Crystal structure of the calcium pump of sarcoplasmic reticulum at 2.6 A resolution”, Nature, 405:647-655.

Bowie, J. U. (2001) “Stabilizing membrane proteins”, Curr Opin Struct Biol, 11:397-402.

Marina, A., Mott, C., Auyzenberg, A., Hendrickson, W., and Waldburger, C. (2001) “Structural and mutational analysis of the PhoQ histidine kinase catalytic domain, Insight into the reaction mechanism”, J Biol Chem, 276:41182-41190.

Schiller, H., Molsberger, E., Janssen, P., Michel, H., and Reilander, H. (2001) “Solubilization and purification of the human ETB endothelin receptor produced by high-level fermentation in Pichia pastoris”, Receptors Channels, 7:453-469.

Shacham, S., Topf, M., Avisar, N., Glaser, F., Marantz, Y., Bar-Haim, S., Noiman, S., Naor, Z., and Becker, O. M. (2001) “Modeling the 3D structure of GPCRs from sequence”, Med Res Rev, 21:472-483.

Tierney, A. J. (2001) “Structure and function of invertebrate 5-HT receptors: a review”, Comp Biochem Physiol A Mol Integr Physiol, 128:791-804.

Venter, J. C., Adams, M. D., Myers, E. W., Li, P. W., Mural, R. J., et al. (2001) “The sequence of the human genome”, Science, 291:1304-1351.

Wurch, T., and Pauwels, P. J. (2001) “Analytical pharmacology of G protein-coupled receptors by stoichiometric expression of the receptor and G(alpha) protein subunits”, J Pharmacol Toxicol Methods, 45:3-16.

Zhou, Y., Morais-Cabral, J. H., Kaufman, A., and MacKinnon, R. (2001) “Chemistry of ion coordination and hydration revealed by a K+ channel-Fab complex at 2.0 A resolution”, Nature, 414:43-48.

Dutzler, R., Campbell, E. B., Cadene, M., Chait, B. T., and MacKinnon, R. (2002) “X-ray structure of a ClC chloride channel at 3.0 A reveals the molecular basis of anion selectivity”, Nature, 415:287-294.

Hunte, C., and Michel, H. (2002) “Crystallisation of membrane proteins mediated by antibody fragments”, Curr Opin Struct Biol, 12:503.

Okada, T., Fujiyoshi, Y., Silow, M., Navarro, J., Landau, E. M., and Shichida, Y. (2002) “Functional role of internal water molecules in rhodopsin revealed by X-ray crystallography”, Proc Natl Acad Sci USA, 99:5982-5987.

Takeda, S., Kadowaki, S., Haga, T., Takaesu, H. and Mitaku, S. (2002) “Identification of G protein-coupled receptor genes from the human genome sequence”, FEBS Lett, 520:97-101.

Valiyaveetil, F. I., Zhou, Y., and MacKinnon, R. (2002) “Lipids in the structure, folding, and function of the KcsA K+ channel”, Biochemistry, 41:10771-10777.

Weiss, H. M., and Grisshammer, R. (2002) “Purification and characterization of the human adenosine A(2a) receptor functionally expressed in Escherichia coli”, Eur J Biochem, 269:82-92.

### SECOND SET OF EXPERIMENTAL DETAILS

Introduction

High-resolution structural studies of proteins generally require large amounts of pure, properly folded material. Indeed, the advent of gene manipulation techniques for producing recombinant protein in heterologous systems is arguably the most important breakthrough of the last thirty years for structural biology, exceeding even the wonderous developments in synchrotron crystallography [1] and NMR spectroscopy [2]. Bacterial expression systems, primarily based on the gram negative bacterium Escherichia coli, have been by far the most successful for the production of recombinant proteins for structural studies. Of the 14,011 protein structures deposited in the Protein Data Bank with ‘Expression_System’ records in their PDB entries (www.rcsb.org), 90.6% were produced using bacterial hosts. This success arises from several factors including the ease with which such organisms can be genetically manipulated; the thorough understanding of their transcription and translation machinery, which has led to the ability to achieve high levels of protein expression; the rapidity of their growth; and the relatively low cost of their use. Despite these advantages, bacterial systems often fail in their application to the expression of eukaryotic proteins [3, 4]. Failure to achieve acceptable expression often arises from toxicity of the foreign protein or its inability to fold or be targeted properly in the bacterial cell. Such problems inevitably result in low levels of expression or protein mis-folding [3, 4]. Thus, despite drawbacks in efficiency, alternative expression systems based on eukaryotic hosts, have been developed for large scale protein production. These include expression in yeast, insect cells, and mammalian cells.

Expression of mammalian proteins has proven to be particularly challenging in heterologous non-mammalian cell systems. It is difficult to ascribe specific reasons for lower levels of success found for mammalian proteins. However since mammalian proteins evolved in the milieu of mammalian cells, it is understandable that both proper folding and stability depends on their presence in this environment. Although it is routine to use mammalian protein expression systems in functional studies, their application to large scale protein production has been deterred by difficulty in obtaining large amounts of material, rapidly, and at a reasonable cost.

Transfection of mammalian cells leads to cell populations heterogeneous with respect to the amount of protein produced by each cell. Initial heterogeneity primarily arises from differences in the number of plasmids entering each cell in the stage of transient expression [5]. Protein expression levels in transiently transfected mammalian cells peak around 48-72 hours after transfection, and inevitably decline thereafter. Thus, the production of stable transfectants is desirable as a constant source of recombinant protein. Generation of stably producing cell lines requires integration of the expression construct into the genome of the host cell. This leads to additional sources of heterogeneity in expression levels, arising from differences in the number of integrants, and their sites of integration.

Here we describe a system for the selection of highly expressing stable mammalian cells, based on the detection of a co-expressed visible marker, the green fluorescent protein (GFP) [6, 7]. GFP provides an excellent means for cytologically localizing a product from any foreign gene which is fused to the GFP. The fused protein is typically functional and can be localized to its site in the cell through its green fluorescence. GFP has the advantage that it is a clonable marker for use in living tissue. In addition, unlike other bioluminescent reporters, it does not require additional proteins, substrates or co-factors to emit light.

In the GFP system, the coding sequence for the gene of interest is placed under the control of a strong constitutive promoter (such as the promoter element derived from cytomegalovirus, CMV [8]). Downstream, after the termination codon for the gene of interest, an internal ribosome entry site (IRES) [9] is followed by the coding sequence for GFP. Transcription from this construct produces a single bicistronic messenger RNA encoding both genes. The IRES element enables binding of the ribosome at the initiation site of GFP. Thus, two separate proteins—the gene of interest and GFP—are translated from the same message, and expression levels of both proteins are thereby coupled.

This system enables efficient selection of high expressors by monitoring the fluorescence of GFP. Use of fluorescence-activated cell sorting (FACS) [10, 11] technology allows for rapid selection of either clonal or non-clonal populations of highly expressing cells.

Methods and Results

As a test for the applicability of this system to high-level expression of functional proteins, we chose two targets, each presenting different challenges. The first target is the rat serotonin receptor subtype 2c (5HT2c) [12], a G-protein coupled receptor (GPCR). GPCRs are a large family of integral membrane proteins characterized by seven transmembrane spanning helices. GPCRs are notoriously resistant to structural studies, in part due to the difficulty of attaining high-level expression of functional protein [13, 14]. To date only one GPCR, bovine rhodopsin, has yielded a high-resolution structure [15, 16]. Rhodopsin, unlike other GPCRs, is present at high levels in rod cell outer segments where it is naturally expressed. The crystal structure of rhodopsin was determined using material purified from natural sources, rather than with a recombinant expression system. The second target, mouse resistin, is a highly disulfide-linked hormone that is naturally secreted from adipocytes [17, 18]. Attempts at expression of resistin in E. coli, either as soluble protein or refolded from inclusion bodies, does not yield properly folded functional protein [19, 20]. Resistin adopts a complex multimeric structure [21], which inevitably represents a challenge to reproduce with fidelity in heterologous expression hosts.

Expression of 5HT2c

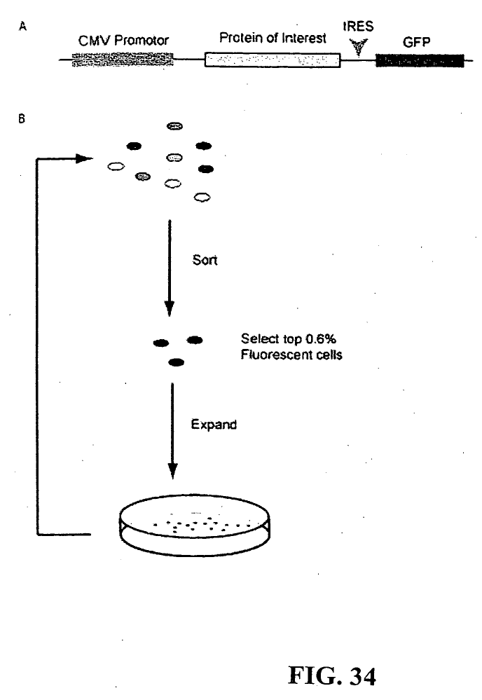

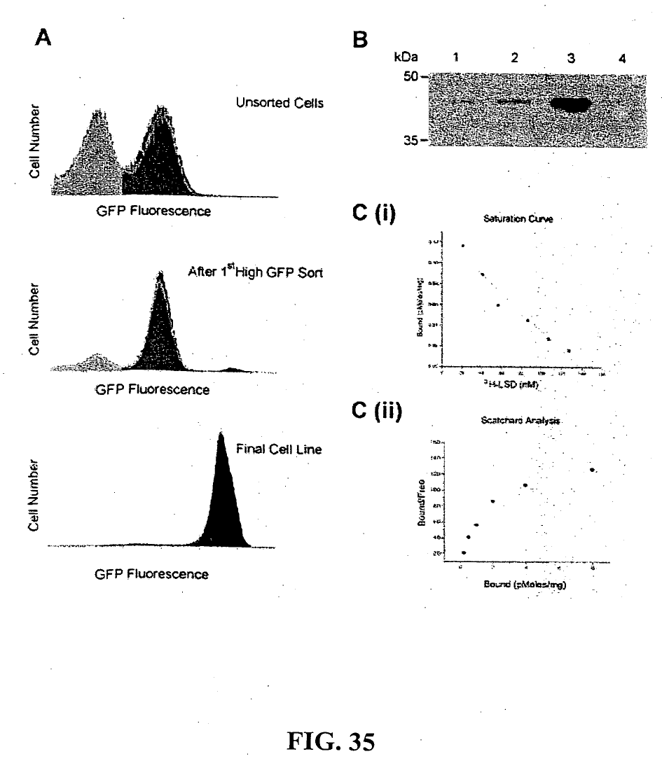

The cDNA for the rat 5HT2c, which encodes a protein of 460 amino acids with three potential glycosylation sites and one palmitoylation site, was inserted into the multiple cloning site of the pCMV-IRES-GFP vector (pFM1.2, FIG. 34A). pFM1.2 carries an antibiotic resistance gene for puromycin under the control of a separate promoter. This construct was transfected into T-antigen transformed human embryonic kidney 293 (HEK-293T) cells using lipofectamine (Invitrogen, Inc.). Stable integrants were selected by growth in puromycin-containining media for a period of approximately three weeks. This resulted in the growth of individual colonies, displaying varying levels of GFP-generated fluorescence. These colonies were pooled, and the resulting cell suspension was then sorted by GFP fluorescence on a Coulter Epics 753 Flow Cytometer. The top 0.1% of the most highly fluorescent cells was separated from the rest, re-plated, and allowed to propogate. This cycle of cell sorting followed by regrowth was repeated five times until a homogeneous level of fluorescence was exhibited by all cells. FIG. 35A shows fluorescence profiles for these cells at different stages of the procedure. FIG. 35B shows quantitative western blots corresponding to whole cell lysates at each of these stages. These data show correlation between GFP-derived fluorescence and the expression level of 5HT2c. Functionality of the expressed protein was assessed by ligand binding analysis of the recombinant protein (FIG. 35C), and reveal saturable binding equivalent to that observed for the naturally produced protein. Furthermore, these data provide a means to quantitate the levels of functional protein, which reach 140-160 pmol/(mg membrane protein), corresponding to approximately 3×10⁶ 5HT2c molecules per cell, or 2.5 mg per 10¹⁰ cells (about 1-5 liters of suspension culture or 2-3 10-layer cell farms [6320 cm²]).

Expression of Resistin

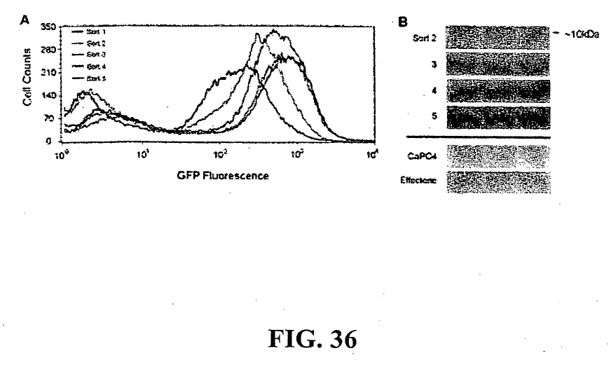

For expression of mouse resistin we inserted its coding sequence into pFM1.1 (FIG. 34A), which is identical to pFM1.2, but lacks an antibiotic selection cassette. Thus, we co-transfected this expression vector with a separate plasmid encoding puromycin resistance, pRSV-puro. Stable HEK-293T cells were obtained by growth in puromycin-containing media, and a FACS-based enrichment protocol similar to that described above was employed. Since resistin is a secreted protein, cell supernatants were used to monitor protein production levels. Similar to 5HT2c, these levels appear to correlate well with the fluorescence of the cells (FIG. 36). Yields on the order of 5 mg/liter were routinely obtained using serum-free media which facilitated purification. Like natural resistin produced by adipocytes and detected in mouse serum, recombinant resistin is hexameric. Physiological insulin clamp studies in mice showed the recombinant protein to function as a potent antagonist of insulin action in the liver [22]. Furthermore, this protein produced crystals that were suitable for diffraction analysis [21].

Discussion

The method described here enables the rapid generation of high-expressing stable mammalian cell lines. The entire procedure, from transfection to obtaining the final cell line, can be accomplished in less than two months time. While this is slow in comparison to bacterial expression methods, it is comparable to the time scale of other widely used methods such as infection of insect cells with recombinant baculovirus, generation of yeast stable integrants, or the production of mammalian cell lines using traditional techniques.

The GFP selection method provides significant advantages in comparison to conventional methods of cell line generation. The isolation of stable integrants in mammalian cells is generally accomplished with the use of antibiotic markers. Expression levels amongst these antibiotic-resistant colonies are highly variable. Traditionally, to screen for high expressing cells, individual colonies are hand-picked, and assayed for their levels of protein production by biochemical methods, usually involving immunological detection. These procedures are time consuming and labor intensive, and thus only a limited number of colonies can be screened. In contrast, the GFP-based selection method described here provides an efficient means for identifying and isolating highly-expressing cells.

The ideal marker for highly expressing cells would be the protein of interest itself. For example, to isolate high expressors of a fluorescent protein one would simply monitor the natural fluorescence. However, for most proteins, no detection method is available. Direct linkages between a protein of interest and a fluorescent marker can easily be constructed as gene fusions, but these are not generally suitable for structural studies. The separation of the fluorescent marker from the protein of interest through the use of an IRES element, enables the production of unmodified protein suitable for structural studies, while maintaining the correlation between expression level and fluorescence within each cell. Furthermore, this separation of target and marker renders the system generally applicable to expression of any protein.

Isolation of the most highly fluorescent cells, corresponding to the highest expressors, can be accomplished in a number of ways. First, visual inspection of fields of colonies enables rapid identification of the most suitable candidates, which can be manually isolated. Alternatively, colonies can be pooled and subjected to FACS analysis. Although, as shown here, the generation of clonal cell lines is not a requirement for achieving high-level expression, the current generation of cell sorters do allow for single cell cloning. Thus, clonal cell lines can be produced with equivalent ease.

Other fluorescent markers, most notably conjugated antibodies, can also be used to select highly expressing cells [23]. Although such markers provide direct correlation to protein expression levels, their use is limited to membrane-attached proteins with extracellular epitopes. Conjugated antibodies cannot enter the cell without a prior lethal permeabilization step, nor are they of use for secreted proteins which are no longer attached to the cells. In contrast, although the GFP selection method correlates fluorescence with expression at the mRNA level, it is not restricted to a limited class of proteins, nor does it depend on the availability of fluorescent markers that bind the protein of interest.

The GFP selection system has potential for future enhancements. These include the possibility for co-expression of multiple genes by constructing bicistronic messages for each, with a different fluorescent protein such as YFP or CFP (Clontech, Inc). This can enable sorting at multiple wavelengths in order to select cells that express all of the proteins highly. Toxic proteins can often be tolerated by cells only under tightly controlled inducible expression. Inducible mammalian expression systems have recently become widely available, and have proven extremely valuable for high level expression of proteins that negatively impact cell viability [24, 25]. Although the system described here provides constitutive expression, in principle it can be modified to provide inducible expression by changing the promoter element.

As structural biology progresses towards the elucidation of increasingly complex macromolecular structures, so too will the need for abundant supplies of the appropriately assembled recombinant molecules. Inevitably, this will lead to an increasing dependence on mammalian expression systems. The GFP selection method presented here addresses one critical step, that of the identification and isolation of highly expressing cells.

### REFERENCES FOR SECOND SET OF EXPERIMENTAL DETAILS

1. Hendrickson, W. A. (2000). Synchrotron crystallography. Trends Biochem Sci 25, 637-643.

2. Wuthrich, K. (2003). NMR Studies of Structure and Function of Biological Macromolecules (Nobel Lecture). Angew Chem Int Ed Engl 42, 3340-3363.
3. Baneyx, F. (1999). Recombinant protein expression in Escherichia coli. Curr Opin Biotechnol 10, 411-421.
4. Makrides, S. C. (1996). Strategies for achieving high-level expression of genes in Escherichia coli. Microbiol Rev 60, 512-538.
5. Geisse, S., Gram, H., Kleuser, B., and Kocher, H. P. (1996). Eukaryotic expression systems: a comparison. Protein Expr Purif 8, 271-282.
6. Chalfie, M. (1995). Green fluorescent protein. Photochem Photobiol 62, 651-656.
7. Tsien, R. Y. (1998). The green fluorescent protein. Annu Rev Biochem 67, 509-544.
8. Thomsen, D. R., Stenberg, R. M., Goins, W. F., and Stinski, M. F. (1984). Promoter-regulatory region of the major immediate early gene of human cytomegalovirus. Proc Natl Acad Sci USA 81, 659-663.
9. Vagner, S., Galy, B., and Pyronnet, S. (2001). Irresistible IRES. Attracting the translation machinery to internal ribosome entry sites. EMBO Rep 2, 893-898.
10. Ibrahim, S. F., and van den Engh, G. (2003). High-speed cell sorting: fundamentals and recent advances. Curr Opin Biotechnol 14, 5-12.
11. Galbraith, D. W., Anderson, M. T., and Herzenberg, L. A. (1999). Flow cytometric analysis and FACS sorting of cells based on GFP accumulation. Methods Cell Biol 58, 315-341.
12. Julius, D., MacDermott, A. B., Axel, R., and Jessell, T. M. (1988). Molecular characterization of a functional cDNA encoding the serotonin 1c receptor. Science 241, 558-564.
13. Sarramegna, V., Talmont, F., Demange, P., and Milon, A. (2003). Heterologous expression of G-protein-coupled receptors: comparison of expression systems fron the standpoint of large-scale production and purification. Cell Mol Life Sci 60, 1529-1546.
14. Grisshammer, R., and Tate, C. G. (1995). Overexpression of integral membrane proteins for structural studies. Q Rev Biophys 28, 315-422.
15. Teller, D. C., Okada, T., Behnke, C. A., Palczewski, K., and Stenkamp, R. E. (2001). Advances in determination of a high-resolution three-dimensional structure of rhodopsin, a model of G-protein-coupled receptors (GPCRs). Biochemistry 40, 7761-7772.
16. Palczewski, K., Kumasaka, T., Hori, T., Behnke, C. A., Motoshima, H., Fox, B. A., Le Trong, I., Teller, D. C., Okada, T., Stenkamp, R. E., Yamamoto, M., and Miyano, M. (2000). Crystal structure of rhodopsin: A G protein-coupled receptor. Science 289, 739-745.
17. Steppan, C. M., Bailey, S. T., Bhat, S., Brown, E. J., Banerjee, R. R., Wright, C. M., Patel, H. R., Ahima, R. S., and Lazar, M. A. (2001). The hormone resistin links obesity to diabetes. Nature 409, 307-312.
18. Holcomb, I. N., Kabakoff, R. C., Chan, B., Baker, T. W., Gurney, A., Henzel, W., Nelson, C., Lowman, H. B., Wright, B. D., Skelton, N. J., Frantz, G. D., Tumas, D. B., Peale, F. V., Jr., Shelton, D. L., and Hebert, C. C. (2000). FIZZ1, a novel cysteine-rich secreted protein associated with pulmonary inflammation, defines a new gene family. Embo J 19, 4046-4055.
19. Juan, C. C., Kan, L. S., Huang, C. C., Chen, S. S., Ho, L. T., and Au, L. C. (2003). Production and characterization of bioactive recombinant resistin in Escherichia coli. J Biotechnol 103, 113-117.
20. Aruna, B., Ghosh, S., Singh, A. K., Mande, S. C., Srinivas, V., Chauhan, R., and Ehtesham, N. Z. (2003). Human recombinant resistin protein displays a tendency to aggregate by forming intermolecular disulfide linkages. Biochemistry 42, 10554-10559.
21. Patel, S. D., Rajala, M. W., Scherer, P. E., and Shapiro, L. (submitted). Disulfide-dependent multimeric assembly of resistin family hormones. Science, submitted.
22. Rajala, M. W., Obici, S., Scherer, P. E., and Rossetti, L. (2003). Adipose-derived resistin and gut-derived resistin-like molecule-beta selectively impair insulin action on glucose production. J Clin Invest 111, 225-230.
23. Mirzabekov, T., Bannert, N., Farzan, M., Hofmann, W., Kolchinsky, P., Wu, L., Wyatt, R., and Sodroski, J. (1999). Enhanced expression, native purification, and characterization of CCR5, a principal HIV-1 coreceptor. J Biol Chem 274, 28745-28750.
24. Reeves, P. J., Callewaert, N., Contreras, R., and Khorana, H. G. (2002). Structure and function in rhodopsin: high-level expression of rhodopsin with restricted and homogeneous N-glycosylation by a tetracycline-inducible N-acetylglucosaminyl-transferase I-negative HEK293S stable mammalian cell line. Proc Natl Acad Sci USA 99, 13419-13424.
25. Reeves, P. J., Kim, J. M., and Khorana, H. G. (2002). Structure and function in rhodopsin: a tetracycline-inducible system in stable mammalian cell lines for high-level expression of opsin mutants. Proc Natl Acad Sci USA 99, 13413-13418.
26. Backstrom, J. R., Westphal, R. S., Canton, H., and Sanders-Bush, E. (1995). Identification of rat serotonin 5-HT2C receptors as glycoproteins containing N-linked oligosaccharides. Mol Brain Res 33, 311-318.

1. A method for producing a membrane-bound protein in high yield, which comprises the steps of
(a) culturing a mammalian cell and progeny thereof having therein an expression vector which coordinately expresses both (i) the membrane-bound protein and (ii) a luminescent protein, under conditions permitting selection of cells expressing the luminescent protein;
(b) selecting cells cultured in step (a) which express a high yield of the luminescent protein so as to thereby select cells expressing a high yield of the membrane-bound protein; and
(c) treating the cells selected in step (b) so as to recover therefrom the membrane-bound protein in high yield.

2. The method of claim 1, wherein the membrane-bound protein is a G protein coupled receptor (GPCR).

3. The method of claim 2, wherein the GPCR is a human GPCR.

4. The method of claim 2, wherein the luminescent protein is green fluorescent protein (GFP).

5. The method of claim 1, further comprising repeating steps (a) and (b) prior to step (c).

6. The method of claim 1, wherein the vector further encodes a protein conferring resistance to an antibiotic, and the conditions permitting selection of cells expressing the luminescent protein encoded by the vector comprise the presence of the antibiotic in a medium in which the cells are cultured.

7. The method of claim 1, wherein the cells selected in step (b) have an average of at least 3 million copies of the membrane-bound protein per cell.

8. The method of claim 7, wherein the cells selected in step (b) have an average of at least 5 million copies of the membrane-bound protein per cell.

9. The method of claim 8, wherein the cells selected in step (b) have an average of at least 10 million copies of the membrane-bound protein per cell.

10-49. (canceled)

50. A method for producing a membrane-bound protein in high yield, which comprises the steps of
(a) culturing a bacterial cell and progeny thereof having therein an expression vector which coordinately expresses both (i) the membrane-bound protein and (ii) a luminescent protein, under conditions permitting selection of cells expressing the luminescent protein;
(b) selecting cells cultured in step (a) which express a high yield of the luminescent protein so as to thereby select cells expressing a high yield of the membrane-bound protein; and
(c) treating the cells selected in step (b) so as to recover therefrom the membrane-bound protein in high yield.

51. A method for expressing a G protein coupled receptor (GPCR) in a bacterial cell comprising culturing a bacterial cell comprising the expression vector of claim 44 or 46.

52. The method of claim 51, wherein the bacterial cell is E. coli.

53. The method of claim 51, wherein the GPCR is a human GPCR.

54-85. (canceled)
